(19) United States
(12) Patent Application Publication
(10) Pub. No.: US 2025/0186979 A1
(43) Pub. Date: Jun. 12, 2025

(54) CATALYST SYSTEM AND PROCESS FOR THE PREPARATION OF ALPHA OLEFINS AND ALPHA OLEFIN-CONTAINING PRODUCTS FROM LONG-CHAIN PARAFFINS

(71) Applicants: PANNON EGYETEM, Veszprém (HU); HUN-REN TERMÉSZETTUDOMÁNYI KUTATÓKÖZPONT, Budapest (HU)

(72) Inventors: Jeno HANCSÓK, Veszprém (HU); Szabina TOMASEK, Veszprém (HU); Norbert MISKOLCZI, Veszprém (HU); Dániel Zoltán HORVÁTH, Rákócziújfalu (HU); József VALYON, Budapest (HU); Róbert TUBA, Veresegyház (HU); Ferenc LÓNYI, Budapest (HU); Márton NAGYHÁZI, Páty (HU); Anna VIKÁR, Érd (HU); Áron BALLA, Százhalombatta (HU)

(21) Appl. No.: 18/839,713

(22) PCT Filed: Feb. 17, 2023
(86) PCT No.: PCT/HU2023/050004

§ 371 Date: Aug. 19, 2024

(30) Foreign Application Priority Data
Feb. 18, 2022 (HU) P2200042
Apr. 14, 2022 (HU) P2200120
Nov. 15, 2022 (HU) P2200450

(51) Int. Cl. B01J 29/74; B01J 31/22; B01J 37/02; B01J 37/08; C07C 5/333

(52) U.S. Cl. CPC B01J 29/7484; B01J 31/2278; B01J 37/0201; B01J 37/088; C07C 5/3337; B01J 2231/543; B01J 2531/821; C07C 2529/74; C07C 2531/22

(57) ABSTRACT

Our invention relates to catalysts and their use in the synthesis and conversion of alpha-olefin-containing products, essentially alpha-olefins, primarily from paraffins of renewable origin, having a carbon number of 11-45, and mixtures thereof, which mixtures contain paraffins having a carbon number of 11-45. The main steps of the process are the heterogeneous catalytic dehydrogenation of paraffin with a new zeolite-supported catalyst based on Pt, Pd or Ni, and then the conversion of the resulting olefin-containing product mixture, containing mainly straight-chain internal monoolefins, to lower homologous alpha-olefins—namely, the homogeneous or heterogeneous catalytic ethenolysis by using a ruthenium complex metathesis catalyst (i.e., metathesis using excess ethylene or ethylene metathesis), and/or tandem isomerization and metathesis reactions by using homogeneous or heterogenized homogeneous ruthenium complex metathesis catalyst in combination with a homogeneous or heterogeneous olefin isomerization catalyst (i.e., isomerization metathesis, hereinafter referred to as ISOMET), and/or tandem isomerization and ethylene metathesis by using a homogeneous or heterogenized homogeneous ruthenium complex metathesis catalyst in combination with homogeneous or heterogeneous olefin isomerization catalyst (i.e., isomerization ethylene metathesis, hereinafter referred to as ethylene ISOMET).

### SUBJECT MATTER OF THE INVENTION

Our invention relates to catalysts and their use in the synthesis and conversion of alpha-olefin-containing products, essentially alpha-olefins, primarily from paraffins of renewable origin, having a carbon number of 11-45, and mixtures thereof, which mixtures contain paraffins having a carbon number of 11-45. The main steps of the process are the heterogeneous catalytic dehydrogenation of paraffin over a new zeolite-supported catalyst based on Pt, Pd or Ni, and then the conversion of the resulting olefin-containing product mixture, containing mainly straight-chain internal monoolefins, to lower homologous alpha-olefins by homogeneous or heterogeneous catalytic ethenolysis using a ruthenium complex metathesis catalyst (i.e., metathesis using excess ethylene, hereinafter referred to as ethylene metathesis), and/or tandem isomerization and metathesis reactions using homogeneous or heterogenized homogeneous ruthenium complex metathesis catalyst in combination with a homogeneous or heterogeneous olefin isomerization catalyst (i.e., isomerization metathesis, hereinafter referred to as ISOMET), and/or tandem isomerization and ethylene metathesis by using a homogeneous or heterogenized homogeneous ruthenium complex metathesis catalyst in combination with homogeneous or heterogeneous olefin isomerization catalyst (i.e., isomerization ethylene metathesis, hereinafter referred to as ethylene ISOMET).

### BACKGROUND OF THE INVENTION

Linear alpha-olefins are known to have a wide range of industrial applications, they can be used for example as motor fuel components or as petrochemical intermediates. One example of them is propylene (propene), which is a starting material or an intermediate in the production of many compounds, namely, an important raw material for the plastics industry.

The industrial production of linear alpha-olefins is largely carried out by ethylene oligomerization, or by extraction from the product mixture of the Fischer-Tropsch synthesis. Relatively small amount is produced via dehydrating alcohols, too. Formerly, terminal olefins were also isolated from the cracking products of paraffins. The disadvantage of the ethylene polymerization process is that it uses ethylene produced purely from crude oil and that only alpha-olefins with even carbon numbers are formed. Therefore, alpha-olefins containing odd number of carbon atoms are almost unaffordably expensive.

Internal olefins used to be produced by chlorination/dehydrochlorination of paraffins. Today, the catalytic paraffin dehydrogenation is the predominant process. U.S. Pat. No. 3,647,906 and patent application with publication number US 2002193649 describe the production of alpha-olefins by the dehydrogenation of paraffins and the subsequent ethenolysis of the resulting material. For dehydrogenation, essentially the PACOL process—described below—is used, and ethenolysis is performed with an oxide-supported rhenium or tungsten catalyst. The process according to our invention differs from the said application in all its essential features: for dehydrogenation we use a more active and selective zeolite-supported metal catalyst than the catalysts according to the cited invention; as well as for ethenolysis a new homogeneous and heterogenized ruthenium complex catalyst is applied. Our process is particularly advantageous for the conversion paraffins of renewable source to alpha-olefins.

In industrial practice, the so-called PACOL process is used to dehydrogenate paraffins to monoolefins. The PACOL process is part of the inventions relating to the value-added conversion of paraffin originating from vegetable oil, and of paraffin produced by the Fischer-Tropsch process as well. There are examples for this, e.g., in the patent documents having a publication number of US 2015/0148561, U.S. Pat. No. 7,737,312, and US 2002/0193649.

The catalyst used in the PACOL process is aluminum oxide doped with platinum or platinum and rhenium, modified with tin, sulfur or alkali metal. Between 1970 and 1987, UOP (Universal Oil Products Company, USA) protected its process with 27 patents. Monoolefin selectivity of 90% was achieved beside the formation of dienes and aromatics. The disadvantage of aluminum oxide-supported metal catalysts used in dehydrogenation is that they can also exhibit aromatizing, skeletal isomerization and cracking. In the catalytic cracking reaction, alpha-olefins are formed in addition to shorter-chain paraffins, but the cracking is accompanied by coke formation and a relatively rapid loss of catalytic activity.

For example, the catalytic dehydrogenation of paraffins is described in the patent application with publication number CN113908880, which discloses zeolite-supported platinum catalysts, preferably bimetallic catalysts, where the other metal besides platinum (Pt) is Zn, Ga, Ce, Fe or Sn. The Na-ZSM-5 zeolite may be impregnated with a solution prepared from a water-soluble Pt salt, while in the case of a bimetallic catalyst additionally with a water-soluble salt of another metal, usually chloride salt, and the salt(s) is/are then removed/decomposed by calcination. From the metals their oxide is formed, which is bound to the zeolite. It is known that the acidic nature of zeolite is unfavorable for the selectivity of olefin formation and catalyst deactivation—as are the Pt particles of a few micrometers in size, due to their alkane hydrogenolysis activity. To mitigate the disadvantages of the unfavorable catalyst property, i.e., the acidity of the zeolite; the process described in CN113908880 starts from zeolites with a high Si/Al ratio (100-500). Positively charged Pt atoms or clusters are stabilized by binding to the zeolite framework, which is negatively charged due to the framework aluminum content. Under the reductive conditions of dehydrogenation, Pt loses its positive charge and the metal particles in the aluminum-poor zeolite framework easily migrate, aggregate, and form large aggregates on the outer surface of the zeolite crystallites. At the same time, the hydrogen reduction of the positively charged platinum or platinum oxide—bound to the negative charge of the zeolite framework—produces Bronsted acid sites in the zeolite. This change that is unfavorable for the dehydrogenation selectivity cannot be avoided by the process described in publication CN113908880.

An example for the production of alpha-olefins by pure catalytic cracking is described by the patent application having the publication number of WO 01/46340. Disadvantageously, the cracking process also produces branched olefin, which is not a suitable reactant for producing linear alkylbenzene sulfonate detergent. It was found that the preferred paraffin dehydrogenation catalysts were supported metal catalysts that are nearly neutral regarding their acid-base properties. Among the supported noble metal catalysts, the more stable ones turned to be those containing the metal—which is active in the dehydrogenation reaction—at a high dispersity on a neutral support. (Songbo He et al.: Industrial development of long chain paraffin (n-C10-C13) dehydrogenation catalysts and the deactivation characterization. Chemical Engineering Journal 275 (2015) 298-304.)

The above-mentioned U.S. Pat. No. 3,647,906 and US 2002193649 patent publications claim the use of oxide-supported rhenium or tungsten catalyst for the ethenolysis of basically internal olefins, which are produced by alkane dehydrogenation. The drawback of these processes is that these catalysts are highly sensitive to the presence of diolefins, in particular conjugated dienes, in the reactant olefin mixture. Prior to the ethenolysis reaction, the concentration of diolefins must be reduced below 1 wt % and the concentration of conjugated dienes below 100 ppm. Another disadvantage is that catalysts lose their activity if the reactant mixture contains even trace amounts of water or oxygen. Their alpha-olefin selectivity is impaired by the fact that the catalysts also show activity in double bond isomerization.

### A BRIEF DESCRIPTION OF THE INVENTION

The objective of the present invention is to develop a new, efficient process for the industrial preparation of linear alpha-olefins using novel catalysts. The starting material for the process according to the invention may be a paraffin, preferably a paraffin of renewable origin having a carbon number of 11-45, or a mixture comprising paraffins having a carbon number of 11-45, or internal monoolefin having a carbon number of 11-45, or a mixture comprising internal monoolefins having a carbon number of 11-45.

According to one aspect of the present invention—i.e., to overcome the drawbacks of the known processes used for the dehydrogenation of paraffins to monoolefins—a new process has been developed for the preparation and use of new catalysts for the dehydrogenation of long-chain normal paraffins that are more active and more selective in the preparation of monoolefins than the known catalysts.

According to another aspect of the process of the invention the disadvantages of the metathesis catalysts—described in the above-mentioned inventions—are eliminated by using novel homogeneous or heterogenized homogeneous ruthenium complex catalysts for the production of alpha-olefins from internal monoolefin, preferably having 11-45 carbon atoms, or from a mixture, comprising internal monoolefins having 11-45 carbon atoms. The new catalysts are more active than the known catalysts and less sensitive to diene, water, and oxygen contamination of the reactant mixture, and show higher functional group tolerance.

The subject matter of the invention relates to a process for preparing linear alpha-olefins having a carbon number of 3-42, or products containing linear alpha-olefins, having a carbon number of 3-42, which process comprises the following steps a) and b):
a) paraffin having a carbon number of 11-45, or a paraffin mixture containing paraffins having a carbon number of 11-45 is dehydrogenated using a zeolite-supported heterogeneous Pt, Pd or Ni catalyst, which catalyst is characterized by that
   it contains a medium pore-sized zeolite as a support, in which the zeolite Si/Al ratio is 2-250, and which has Pt, Pd or Ni metal introduced into the zeolite support with a molar amount not exceeding the framework aluminum content of the zeolite support, at a dispersity higher than 10% and in the form of neutral metal atoms or neutral metal nanoparticles;
and/or
   it is produced by the following process steps:
   (i) a Pt, Pd or Ni salt, preferably Pt, Pd or Ni nitrate, acetate, hydroxide, or a Pt, Pd or Ni amine complex, of a molar amount not exceeding the framework aluminum content of the zeolite, is introduced by ion exchange or impregnation into the Na, K, or ammonium ion form of a medium pore-sized zeolite with a Si/Al ratio of 2-250,
   (ii) the material obtained in step (i) is calcined at 300-500° C.
   (iii) the material calcined in step (ii) is reduced by hydrogen at 300-500° C.,
   (iv) the material reduced in step (iii) is ion-exchanged with a Na or K salt solution, or
   it is reacted in solid phase with Na or K salt in an amount equivalent to the lattice aluminum content of the zeolite, and the salt is then decomposed by heat treatment;
and
b) the internal monoolefin content of the mixture obtained in step a) is converted to alpha-olefins,
   using a homogeneous or heterogenized homogeneous ruthenium complex metathesis catalyst in ethylene metathesis (ethenolysis) reaction, and/or
   using a homogeneous or heterogenized homogeneous ruthenium complex metathesis catalyst in combination with a homogeneous or heterogeneous olefin isomerization catalyst in an isomerization metathesis (ISOMET) reaction, and/or
   using a homogeneous or heterogenized homogeneous ruthenium complex metathesis catalyst in combination with a homogeneous or heterogeneous olefin isomerization catalyst in an ethylene isomerization metathesis (ethylene ISOMET) reaction;
or a process comprising the following step b):
b) an internal monoolefin, having a carbon number of 11-45, or the internal monoolefin content of a mixture containing internal monoolefins having a carbon number of 11-45 is converted to alpha-olefins,
   using a homogeneous or heterogenized homogeneous ruthenium complex metathesis catalyst in ethylene metathesis (ethenolysis) reaction, and/or
   using a homogeneous or heterogenized homogeneous ruthenium complex metathesis catalyst in combination with a homogeneous or heterogeneous olefin isomerization catalyst in an isomerization metathesis (ISOMET) reaction, and/or
   using a homogeneous or heterogenized homogeneous ruthenium complex metathesis catalyst in combination with a homogeneous or heterogeneous olefin isomerization catalyst in an ethylene isomerization metathesis (ethylene ISOMET) reaction;
where the ruthenium complex in the homogeneous or heterogenized homogeneous ruthenium complex metathesis catalyst used in step b) is of formula I, II, III or IV,

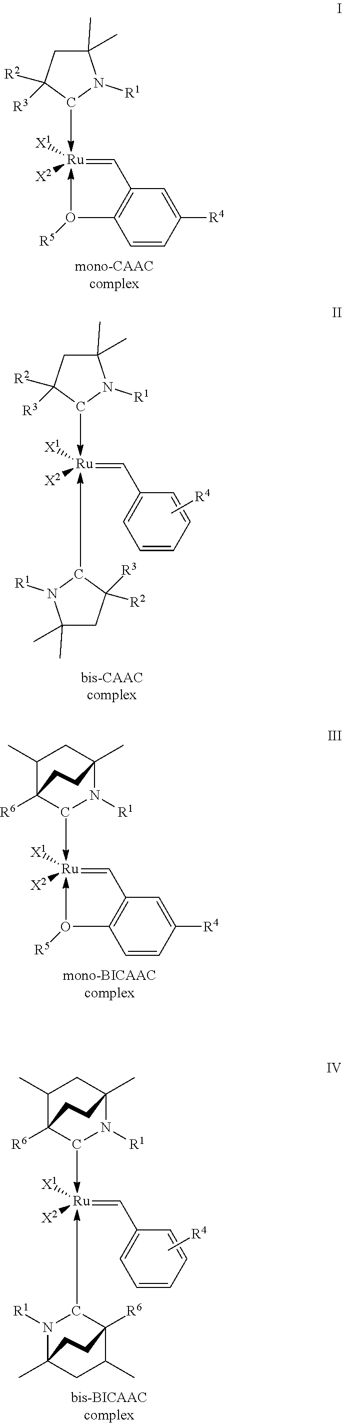

The invention also relates to zeolite-supported Pt, Pd or Ni catalysts used in step a) of the above process.

The invention also relates to ruthenium complexes of formula (III), i.e., ionic or ion-forming group functionalized bicyclic (alkyl)(amino)carbene complex (BICAAC) and/or of formula (IV), i.e., bis-[bicyclic (alkyl)(amino)carbene]complex (bis-BICAAC) used as catalysts in step b) of the above process; as well as ionic or ion-forming group-functionalized, heterogenized homogeneous, ruthenium complex metathesis catalyst of formula (I), i.e., cyclic (alkyl)(amino)carbene complex (CAAC) and/or of formula (II), i.e., bis-[cyclic (alkyl)(amino)carbene]complex (bis-CAAC) and/or of formula (III), i.e., bicyclic (alkyl)(amino)carbene complex (BICAAC) and/or of formula (IV), i.e., bis-[bicyclic (alkyl)(amino)carbene]complex (bis-BICAAC).

The invention further relates to a process for the preparation of olefins or olefin-containing products from paraffin or from a paraffin mixture, which process comprises the dehydrogenation of paraffin using a zeolite-supported heterogeneous Pt, Pd or Ni catalyst as described in step a) above.

### DETAILED DESCRIPTION OF THE INVENTION

In step a), the dehydrogenation reaction according to equation (1) is performed using a new zeolite-supported Pt, Pd or Ni metal catalyst.

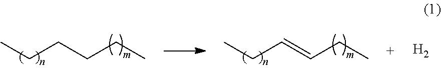

where n+m is at least 7 and at most 41, and n or m can have a minimum value of zero as well.

The probability of alpha-olefin formation during catalytic dehydrogenation is small, but if an alpha-olefin is formed, it is not converted to a new product during ethenolysis. The straight-chain olefin-containing product mixture obtained by dehydrogenation is reacted in step b) of the process with a ruthenium complex metathesis catalyst with more advantageous properties than known catalysts, in homogeneous or heterogeneous catalytic ethylene metathesis reaction (ethenolysis) to produce a product mixture of olefins containing shorter-chain alpha olefins in the reaction according to equation (2).

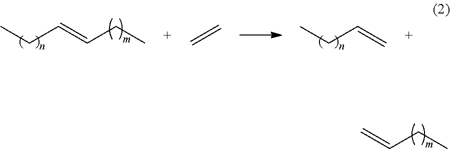

The alpha-olefins formed during ethenolysis are optionally further converted by the ethylene ISOMET process according to equation (4).

The ruthenium complexes used to catalyze the metathesis may form a homogeneous phase with the reaction mixture or may be solid-supported heterogenized ruthenium complex catalysts. In addition to the ruthenium complex metathesis catalyst, the ISOMET reaction system also contains a catalyst for the isomerization of olefin double bonds, which may be a homogeneous (e.g., ruthenium hydride) and/or heterogeneous (e.g. H-BEA zeolite) catalyst. The ethenolysis-active homogeneous ruthenium complex metathesis catalyst is preferably heterogenized by attaching it to a support that actively catalyzes the isomerization of the double bond. On the catalyst surface, the ISOMET conversion of alpha-olefins takes place according to equation (3a, isomerization) and (3b, metathesis) to yield shorter and longer chain olefins, respectively.

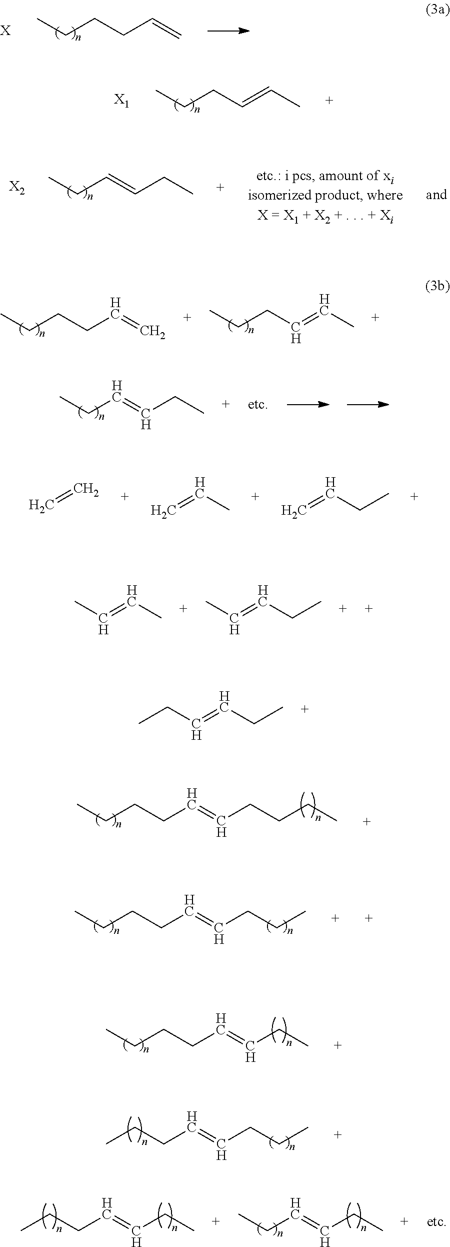

A special case of ISOMET reactions according to equations (3a) and (3b) is the ethylene ISOMET conversion according to equation (4) in the presence of excess ethylene:

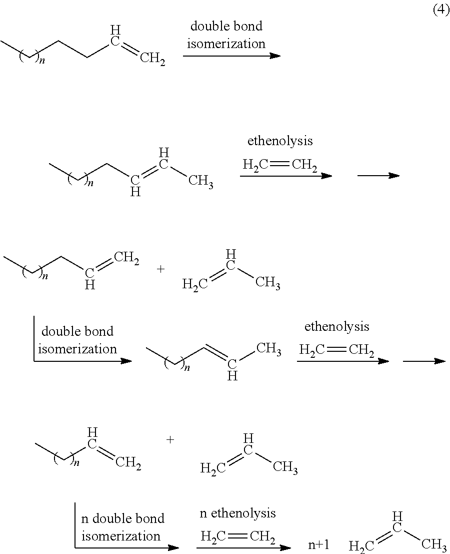

whereby the compounds present in the mixture having unsaturation at different positions are eventually converted to propylene. From equation (4), it can generally be concluded that N−2 molecules of propylene form from a terminal (alpha-)olefin, containing N carbon atoms, by a stepwise double bond isomerization and ethenolysis (the latter requires N−2 molecules of ethylene). Since the metathesis of olefins is an equilibrium reaction, the equilibrium is shifted towards propylene formation by using ethylene excess. For the metathesis reaction, a long, straight-chain (>C₁₁) monoolefin other than alpha-olefin is used.

According to the present invention, in step a) of the process, novel zeolite-supported Pt-, Pd- or Ni-based dehydrogenation catalysts are used.

The supported zeolites are composed of interconnected SiO_{4/2} and AlO_{4/2} tetrahedra, where the subscript 4/2 indicates that each oxygen is bonded to two Si atoms or to one Si and one Al atom (T atom). So far, 247 skeletal structures have been described. Zeolites are characterized, among other things, by their channel (microporous) systems defined by their crystal structure. A distinction is made among zeolites based on pore sizes, such as small pores, medium pores, large pores and extra-large pores. The channel system is characterized by the size of the largest pore opening. The channel openings of zeolites are bounded by tetrahedra, linked through shared oxygen atom. The size of the boundary ring is essentially determined by the number of T atoms (or the equivalent number of oxygen atoms). The ring is usually elliptical in shape, only slightly different from the circle shape.

The largest pore openings of small-pore zeolites are bounded by up to 8 tetrahedra, i.e., 8 T atoms and 8 oxygen atoms, i.e., the pore openings are bounded by an 8-membered ring (0.4 nm). The largest pore openings of medium-pore zeolites are bounded by 10 tetrahedra (0.55 nm). The largest pore openings of the large-pore zeolites are bounded by 12 tetrahedra (0.75 nm), the extra-large pore zeolites by more than 12 tetrahedra (>0.75 nm).

Medium pore-size zeolites with medium pore openings bounded by a ring containing 10 oxygen atoms with MFI, TON and IMF skeletal structures were chosen as support. Among the zeolites with MFI structure, ZSM-5 is preferred, among the zeolites with TON structure, ZSM-22 is preferred, and among the zeolites with IMF structure, IM-5 is preferred. The zeolite Si/Al ratio is between 2 and 250, preferably between 5 and 150, most preferably between 10 and 80. The zeolite is preferably ZSM-5 and ZSM-22 zeolites, most preferably ZSM-22 zeolite. Detailed information on zeolites can be found, for example, on the website of the International Zeolite Association (https://europe.iza-structure.org/IZA-SC/ftc_table.php). The pore system of the ZSM-5 zeolite is three-dimensional. The maximum sizes of the elliptical openings of channels are 0.56 and 0.57 nm. IM-5 zeolite is characterized by two two-dimensional pore systems interconnected by pores. The three types of pores have maximum diameter of 0.54, 0.56, and 0.59 nm. In the one-dimensional pore system of the ZSM-22 zeolite, the largest pore opening diameter is 0.57 nm.

The catalyst was developed taking into account factors that are affecting the selectivity of catalytic paraffin hydroconversion. In hydroconversion, the acidic form of the zeolite is active. It is known that shape-selective catalytic effects prevail in micropores bounded by a 10-membered ring. On the ZSM-5-type bifunctional catalyst(s), mainly single-branched paraffins are formed and cracked, whereas on the ZSM-22 zeolite, branched carbon chains cannot be formed and therefore cracking is a slow process. In the narrow zeolite pores, comparable to the critical molecular size of paraffins (˜0.43 nm), the accumulation of carbon deposits is inhibited. This contributes to the slow deactivation of the catalyst. Cracking is reduced if the residence time of the reaction intermediates in the pores is shorter, that is, the smaller size of the zeolite crystallite is more favorable for double bond isomerization selectivity. The larger metal particles are the active ones in the hydrogenolysis of paraffins. Hydrogenolysis and methane formation are reduced if the catalyst contains nanosized metal particles.

A dehydrogenation catalyst is expected to have low isomerization, cracking, olefin polymerization and hydrogenation activity; and expected to have high dehydrogenation activity and selectivity. This purpose is primarily served by the use of medium pore-size zeolite supports. The zeolite supports used in dehydrogenation should not be acidic in character and the active metal is preferably stabilized in a form of highly dispersed zero-charge metal atom or nanocluster.

Dispersity means the surface metal content of the supported catalyst metal particles as a percentage of the total metal content. The higher the dispersity, the smaller the particles. By high dispersity, we preferably mean a dispersity of at least 10%. To determine dispersity, the total metal content of the catalyst is measured by chemical analysis and the number of surface atoms is determined by chemisorption using, e.g., hydrogen or carbon monoxide as adsorbate. X-ray diffraction or electron microscopy images provide information on the particle size and can be used to infer the dispersity. The metal dispersity in zeolite can be of a few 10%.

The dehydrogenation catalyst is prepared in the following steps:

The medium-pore-size zeolite is converted by exhaustive ion exchange to its ammonium ion form or alkali metal form, preferably Na or K form, most preferably NH₄⁺ form. (i) Into the alkali metal or ammonium form of the zeolite a precursor of the metal (Me, where Me is Pt, Pd or Ni), preferably the nitrate, acetate, hydroxide or amine complex of the metal is introduced by ion exchange or impregnation. (ii) The metal-containing composition is calcined under slow heating at 300-500° C. in an oxygen or oxygen/inert gas stream. (iii) The calcined sample is reduced with hydrogen at a temperature, at which the complete reduction of the metal ions already occurs, typically at 300-500° C. Finally, (iv) the resulting Me/Na, H-zeolite, Me/K, H-zeolite, or Me/H-zeolite preparation is treated by acidity neutralizing ion exchange with a solution containing sodium or potassium cations, or the zeolite preparation and a sodium or potassium salt, for example sodium or potassium chloride, preferably sodium or potassium nitrate, acetate or hydroxide is reacted in a mixture of the salt and of the zeolite preparation, in the solid phase, and the salt is then decomposed by heat treatment.

It is known that the reduction of the metal cation—that compensates the lattice charge—generates acidic hydroxyl groups that take over the compensation role from the metal ion, i.e., the compensation of the negative charge of the zeolite framework, because acidity was not advantageous for the selective dehydrogenation. Process step (iv) serves to neutralize acid sites.

The invention also relates to zeolite-supported Pt, Pd or Ni catalysts as described above and/or obtained by the above process, comprising a medium pore-size zeolite as support, preferably MFI, TON or IMF framework, in particular, a zeolite of the type ZSM-5, ZSM-22 or IM-5, wherein the zeolite Si/Al ratio is 2-250, preferably 5-150, most preferably 10-80, and it contains Pt, Pd or Ni metal introduced into the zeolite support with a molar amount not exceeding the framework aluminium content of the zeolite, at a dispersity of higher than 10% and in the form of zero-charged metal atoms or of zero-charged metal nanoparticles. In the novel, zeolite-supported Pt, Pd or Ni catalysts, used in step a) according to the invention, the dispersity of the metal is higher than in the prior art zeolite-supported metal catalysts. In addition, no metal-catalyzed alkane hydrogenolysis or adverse acid-catalyzed reactions occur on the catalysts. Thus, the catalysts according to the invention exhibit advantageous dehydrogenation activity, stability and selectivity.

The dehydrogenation step a) is preferably carried out in a continuous-flow tubular reactor. To limit undesired polymerization of olefins, the dehydrogenation reaction is performed in hydrogen at 0.5-1.0 bar overpressure at 350-500° C., with a relatively short space time. The space velocity of the fed paraffin liquid (Liquid Hourly Space Velocity, LHSV, the mass flow of the fed liquid in relation to the mass of the catalyst) is 5-25 h⁻¹, preferably 15-25 h⁻¹, most preferably 20 h⁻¹. The space velocity of hydrogen (Gas Hourly Space Velocity of Hydrogen, GHSV, the volume flow rate of the feed gas calculated for normal conditions in relation to the volume of the active catalyst bed) is 8000-15000 h⁻¹, preferably 10000-15000 h⁻¹, most preferably 12000 h⁻¹. Before starting the reaction, the catalyst is activated in a stream of hydrogen under the reaction conditions. In case of slow catalyst deactivation, the reaction temperature can be increased within the specified temperature range to keep the conversion constant.

The process according to the invention relates to the conversion of straight-chain paraffins with carbon number of 11-45 or of paraffin mixtures into straight-chain alpha-olefins. The raw material for the process may be paraffin product from the petroleum industry or preferably, paraffin produced from a renewable raw material. The latter include Fischer-Tropsch wax (FT wax; commonly called as FT wax or its technically correct name is high-molecular-weight/high carbon number paraffin mixture) produced from bio-derived synthesis gas, or paraffin hydrocarbons produced from vegetable oils, fats or fatty acids, or from their other derivatives, etc. A common advantage of bioparaffins for the catalytic process is that they practically do not contain sulfur compounds.

Paraffins are prepared from fatty acids and fatty acid derivatives (e.g., triglycerides) by hydrodeoxygenation (HDO) reaction. In the HDO reaction, the double bonds—that are often present in the long carbon chain of fatty acids—saturate, therefore these paraffins must be dehydrogenated before ethenolysis. For our process, paraffins prepared from fatty acids and/or fatty acid derivatives and FT wax can be used alone or in their mixtures. The amount and composition of the 11-45 carbon number fraction of the FT wax depends mainly on the catalyst used in the FT process and on the reaction temperature. On a supported Fe catalyst and at high temperature (Fe-HTFT), about 10% by weight of the product mixture produced is a fraction containing straight-chain molecules having 11-45 carbon atoms. The olefin content of the fraction can reach 65% and the oxygenate (mainly alcohol) content can be 7-8%. Because of the high oxygenate content, this fraction should be hydrodeoxygenated, possibly using a mixture of triglycerides and/or fatty acids. The paraffin product of the HDO process can be one of the preferred raw materials for the process according to our invention. The product mixture of low-temperature FT processes (Co-LTFT, Fe-LTFT) containing molecules with 11-45 carbon atoms can reach 65-70% of the FT product mixture weight. The fraction is mainly straight-chain paraffin; its olefin and oxygenate content is low: 1-10% and <0.5%, respectively. The product mixture containing molecules having 11-45 carbon atoms, produced by the low temperature FT process—in case they are produced from biosolids or synthesis gas or from other renewable feedstocks—can also be a preferred raw material for our dehydrogenation process, either alone or in a mixture with other bioparaffin feedstock streams. Equally advantageous raw materials may include, for example, e-paraffins (individual paraffins or mixture of paraffins produced from the mixture of (i) hydrogen—which is formed with the aid of electricity from renewable energy sources—and (ii) CO₂ and/or (iii) carbon monoxide—trapped from the air or derived from other sustainable sources) or paraffin mixtures from, for example, the hydrogenated cracked waste polyethylene fractions.

A product mixture—containing an increased amount of olefin, obtained from long-chain paraffin or paraffinic mixture by dehydrogenation—can be a raw material for ethenolysis according to equation (2) for the production of alpha-olefins. One advantage of the process compared to the alpha-olefin production by cracking is that the reaction produces two alpha-olefin molecules of the same or different chain lengths from a single long-chain olefin molecule, and that it also produces alpha-olefins with odd numbers of carbon atoms. When a paraffin is dehydrogenated, diolefins are formed in addition to monoolefins. The latter must be selectively reduced to monoolefins before olefins can be used for aromatic alkylation, as they can be linked to two aromatic rings. This is done by a process patented by UOP known as DeFine process using aluminum oxide-supported Ni catalyst. The advantage of ethenolysis is that alpha-olefins are formed also from diolefins and there is no need for selective catalytic hydrogenation of the olefin product mixture to monoolefins prior to alkylation. An additional advantage is that ethenolysis is a more energetically favorable, low-temperature reaction than both the DeFine process and cracking, without the rapid coke formation and catalyst ageing (loss of activity and selectivity) that usually occurs during cracking. The atomic recovery of the ethenolysis reaction is close to 100%, i.e., it is essentially a by-product-free process.

According to another aspect of our invention, the ethenolysis process of step b) described by equation (2), the ISOMET or ethylene ISOMET process described by equations (3) or (4), respectively, is performed using new homogeneous or heterogenized homogeneous catalyst of formula (I), (II), (III) or (IV). A heterogenized homogeneous catalyst combines the advantages of homogeneous and heterogeneous catalysts. The homogeneous catalysts are more active and selective than the supported molybdenum, tungsten or rhenium oxide catalysts commonly used in metathesis reactions, while the heterogenized homogeneous catalysts are similar to oxide catalysts in that they can be easily separated from the reaction mixture and reused.

A very similar structure to our catalyst system used in step b) was described in patent application having a publication number of WO2022008679 A1. The structure of the BICAAC carbene ligand described therein, however, is not suitable to catalyze the reaction of step b) of the present invention according to the results of our experiments, since all of the compounds specifically described on pages 24-25 contain isopropyl substituents on the aromatic ring attached to nitrogen at the ortho positions relative to the nitrogen.

The prior art patent application having a publication number of WO2020109217A2 also describes ruthenium complex metathesis catalysts, but the structure of these compounds differ from the ruthenium complexes of the invention. Additionally, the disclosure of WO2020109217A2 and the structure of the complexes described therein do not imply the parameters necessary for the operation of step b) of the process of our invention.

According to a further aspect of the present invention it relates to monocyclic (alkyl)(amino)carbene complexes (CAAC and bis-CAAC according to formula (I) and (II), respectively) and bicyclic (alkyl)(amino)carbene complexes (BICAAC and bis-BICAAC according to formula (III) and (IV), respectively) as homogenous ruthenium-based catalysts having ionic or ion-forming groups—for example, quaternary ammonium ion, amine, carboxylic acid, carboxylate, sulfonic acid and sulfonic acid salt groups. The present invention also relates to the preparation, heterogenization of the said complexes, and to the use of the homogenous or heterogenized homogenous catalysts in the ethylene metathesis reaction of long-chain olefins. The catalysts are suitable for the production of a product mixture containing alpha-olefins having 3-42 carbon atoms, mostly having 10-20 carbon atoms, by ethylene metathesis of monoolefins having 11-45 carbon atoms. The invention further relates to the use of the homogeneous or heterogeneous metathesis catalysts in combination with a known homogeneous or heterogeneous olefin double bond isomerization catalyst, for the isomerization metathesis (ISOMET) of olefinic compounds of arbitrary composition, and for their isomerization metathesis with ethylene, preferably with excess ethylene (ethylene ISOMET).

The selection of the substituents of the catalyst systems used is a significant factor in step b). The complexes with the appropriate structure are those of formula I, II, III or IV,

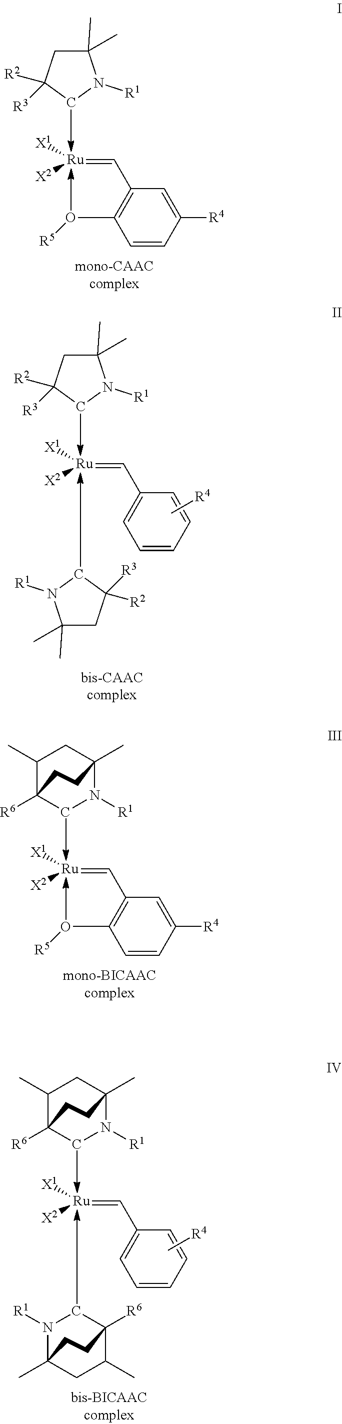

wherein,
the chemical valency ligands X¹ and X² linked to ruthenium are, independently from each other halogen, nitrite, nitrate, sulfate, carboxylate, sulfonate, alkoxy, or alkylthio, or X¹ and X² are heteroatoms, linked to each other through an organic molecular part (for example, X¹ and X² represent O and form together a through a benzene ring catecholate group, or X¹ and X² are S and form together through a benzene ring a catecho-dithiolate group); X¹ and X² represent, according to an embodiment, a halogen atom, in particular chlorine;
R¹ represents aryl, heteroaryl or alkyl optionally substituted by one or more substituents, where the substituents are independently selected from alkyl, aryl, heteroaryl, amino, alkylamino, dialkylamino, quaternary ammonium ion, nitro, sulfone, sulfonic acid, sulfonic acid derivative, ketone, carboxylic acid, carboxylic acid derivative, alkoxy, preferably an electron-donating group such as dimethylamino or trimethylammonium ion, with the proviso, that when R¹ is aryl or heteroaryl, in the case of a BICAAC complex of formula (III) or (IV) the substituent in the ortho position relative to the nitrogen atom is an alkyl containing at most 1-2 carbon atom(s), otherwise the complex shows no metathesis or ISOMET activity;
according to one embodiment, R¹ represents, an aryl group optionally substituted by one or more substituents, particularly, a phenyl group optionally substituted by one or more substituents, particularly phenyl optionally substituted by one or more substituents selected from alkyl (preferably alkyl having 1-2 carbon atoms), amino, alkylamino having 1-6 carbon atoms, preferably having 1-2 carbon atoms, dialkylamino having 1-6 carbon atoms, preferably having 1-2 carbon atoms, and quaternary ammonium ion, preferably 2,6-dimethyl-4-dimethylamino-phenyl, or 2,6-dimethyl-4-trimethylammonium-phenyl;
R² and R³ represent independently alkyl, aryl or heteroaryl, which may be optionally substituted by one or more substituents selected from alkyl, aryl, heteroaryl, amino, alkylamino, dialkylamino, quaternary ammonium ion, nitro, sulfone, sulfonic acid, sulfonic acid derivative, ketone, carboxylic acid, carboxylic acid derivative, alkoxy, preferably an electron-donating group, or R² and R³ form together with the carbon atom to which they are linked a cycloalkane ring having 3-12 carbon atoms,
R² and R³ represent in one embodiment alkyl or aryl, such as methyl or phenyl;
R⁴ represents hydrogen, alkyl group, nitro group, carboxylic acid, carboxylic acid derivative, sulfonic acid, sulfonic acid derivative, aryl group, heteroaryl, perfluoroalkyl, perfluoroaryl, sulfone, ketone, preferably an electron-withdrawing group such as a perfluoroalkyl group, or nitro group, R⁴ represents hydrogen according to one embodiment;
R⁵ represents an alkyl group such as isopropyl;
R⁶ represents hydrogen or an alkyl group, or a group linked via a linker of 1-24 carbon atoms, wherein the group is selected from aryl, heteroaryl, amino, alkylamino, dialkylamino, ketone, ether, hydroxyl, thioether, thiol, carboxylic acid derivative, sulfonic acid derivative, silicon-containing group, or a ligand with a metal coordinating property, such as amine, imine, sulfide, sulfoxide, phosphine, ether, nitrile or isonitrile; a special case of the latter, where said group at the R⁶ position is attached to other variable groups of the molecule, for example, in the case of a bis-BICAAC complex of formula IV, to the R⁶ group of the other BICAAC ligand;
R⁶ represents in one embodiment alkyl, for example methyl;
with the proviso that at least one of the groups R¹, R², R³, or R⁶ contains an ionic or an ion-forming group, for example, a quaternary ammonium ion, amine, alkylamino having 1-6 carbon atoms, dialkylamino having 1-6 carbon atoms, carboxylic acid, carboxylate, sulfonic acid or a sulfonic acid salt group.

The invention relates to the ruthenium complexes of formula III or IV, wherein the meaning of X¹, X², R¹, R⁴, R⁵ and R⁶ are defined above.

The preparation of (III) or (IV) complexes containing a BICAAC ligand can be divided into two steps: the preparation of the BICAAC ligand and the complexation of the BICAAC ligand with the ruthenium alkylidene part of the molecule (derived from known ruthenium alkylidene complexes).

The BICAAC ligand can be prepared according to the synthesis method described in the following publication: E. Tomás-Mendivil et al., J. Am. Chem. Soc. 2017, 139, 23, 7753-7756, with minor modifications. A primer amine (R¹—NH₂, practically 2,6-disubstituted aniline) corresponding to the N-substituent (R¹) of the prospective ligand is reacted with 2,4-dimethylcyclohex-3-ene-1-carbaldehyde (also known by its commercial name: Trivertal) in such a way that aldimine is formed. For these reasons, suitable components are molecular sieves that are acidic in character as well as water-binding in nature, and a dry solvent, such as dichloromethane. Imine is formed in 16 hours at room temperature. After isolation, the aldimine is C-alkylated at its alpha-position in a strong base (e.g., lithium diisopropylamide) in an ether type solvent (e.g., tetrahydrofuran) and an aliphatic substituent is introduced into the molecule to form the R⁶ group of the future ligand. The leaving group of the alkylating agent is preferably a halide, or possibly a derivative of a strong organic carboxylic acid or sulfonic acid (e.g., tosylate or triflate). The reaction shall be started by deprotonating the imine at 0° C. and then it has to be stirred at room temperature for four hours, finally the alkylating agent has to be added at 0° C. The reaction usually takes 12 hours when stirred at room temperature. The resulting alkylated imine is then reacted with 5 molar equivalents of hydrogen chloride in anhydrous ether-type solvent in a pressure vessel at 80° C. for 48 hours. The ring-closure reaction results the cationic iminium salt product with 2-azabicyclo[2.2.2]octane ring system. The chloride counterion should preferably be replaced by a non-coordinating counterion (e.g., with the aqueous solution of ammonium tetrafluoroborate to BF₄ anion). The product is a precursor of carbene (HBF₄ salt) which can be purified by recrystallization from diethyl ether.

The ionic or ion-forming group can be placed on either R¹ or R⁶, and the appropriate starting material (amine or alkylating agent) has to be chosen.

The complexation is performed in THF solvent in the presence of a strong base (e.g., lithium hexamethyldisilylazide). The source of Ru-alkylidene is the complex corresponding to the target compound (III or IV), with phosphine or pyridine as an exchangeable ligand. Examples are the Grubbs and Hoveyda-Grubbs type complexes, where tricyclohexylphosphine ligand is exchangeable. Ligand exchange requires a 1.5-fold carbene excess in respect to the leaving ligand. The final complex can be purified by chromatography. The formation of ionic groups from tertiary amine functional groups can be carried out with a strong alkylating agent (e.g., methyl trifluoromethanesulfonate) at −30° C. in dichloromethane solvent. The ionic product is obtained by evaporation.

All of the processes described here require dry and oxygen-free reaction conditions. The products can be processed in air but must be stored in an inert atmosphere.

### Definitions

Alkyl means a chemical group that does not contain atoms other than carbon and hydrogen, and multiple covalent bonds; it contains at least 1 and maximum 12 carbon atoms, and has any degree of branching. This includes cyclic groups, regardless of whether the attachment point is inside or outside the ring. There may by an additional atomic group or function on these groups. Alkyl means, for example, methyl, ethyl, propyl, isopropyl, butyl, t-butyl, cyclopropyl, especially methyl or ethyl groups or cyclopropyl.

Aryl means a chemical group having a cyclic structure in which conjugation occurs, consisting of at least 6 and up to 14 carbon atoms, such as phenyl, naphthyl, anthracil, especially phenyl. There may be an additional atomic group or functional group attached to the aryl group.

Heteroaryl means a chemical group that has a cyclic structure, in which conjugation occurs, containing 5-14 atoms, at least one of which is a heteroatom, such as nitrogen, sulfur, oxygen. Heteroaryl groups are for example thienyl, furyl, pyridyl, naphthyl, indenyl, quinolinyl, especially pyridyl, quinolinyl or thienyl. There may be an additional atomic group or functional group on the heteroaryl group.

Halogen means chlorine, iodine, bromine or fluorine, particularly chlorine.

Carboxylic acid means —COOH group.

A carboxylic acid derivative means —COO-alkyl, where alkyl is defined above, particularly alkyl having 1-6 carbon atoms, or —CO-halogen.

Carboxylate means —COO⁻ group or its salt.

Nitrite means —O—NO group.

Nitrate means —O—NO₂ group.

Alkoxy means —O-alkyl group, where alkyl is as defined above, preferably alkyl having 1-6 carbon atoms.

Alkylthio means —S-alkyl group where alkyl is as defined above, particularly alkyl having 1-6 carbon atoms.

Amino means —NH₂ group.

Alkylamino means an —NH-alkyl group, where alkyl is as defined above, particularly alkyl having 1-6 carbon atoms.

Dialkylamino means an —N-(alkyl)₂ group, where the two alkyl groups are independently alkyl, selected from those given above, particularly alkyl having 1-6 carbon atoms, for example dimethylamino or diethylamino group.

Quaternary ammonium ion is —N⁺(alkyl)₃ or its salt, where the three alkyl groups are independently selected from each other, from the above meanings, particularly alkyl having 1-6 carbon atoms, for example—N⁺(CH₂CH₃)₃ or for example N⁺(CH₂CH₃)₂(CH₃).

Sulfone means —SO₂-alkyl group, where alkyl is defined above, particularly alkyl having 1-6 carbon atoms.

Sulfoxide means —SO-alkyl group, where alkyl is defined above.

Sulfonic acid means —SO₂OH group.

Sulfonate represents —SO₂O⁻ group or its salt.

Sulfonic acid derivative means —SO₂—O-alkyl where alkyl is defined above or —SO₂-halogen.

Sulfate means —OSO₂O⁻ group or its salt.

Ketone means —CO-alkyl group where alkyl is defined above, particularly alkyl having 1-6 carbon atoms.

Carboxylic acid means —COOH group.

A carboxylic acid derivative means —COO-alkyl, where alkyl is defined above, particularly alkyl having 1-6 carbon atoms, or —CO-halogen.

Perflouoroalkyl means an alkyl group where all the hydrogen is fluorine-substituted and where alkyl is defined above.

Perfluoroaryl means an aryl group where all the hydrogens are fluorine-substituted and where aryl is as defined above.

Hydroxyl means —OH group.

Tiol means —SH group.

Ether means —O-alkyl group where alkyl is as defined above, particularly alkyl having1-6 carbon atoms.

Thioether means —S-alkyl group, where alkyl is defined above, in particular alkyl having 1-6 carbon atoms.

Imine means —C(alkyl)=N-alkyl group, where alkyl is independently selected from those given above.

Phosphine means —P(alkyl)₂, wherein the two alkyls are independent from each other, selected from those given above.

Nitrile means —CN group.

Isonitrile means —N⁺≡C⁻.

Silicon-containing group means —Si(alkyl)₃ or —O—Si(alkyl)₃ group, where alkyl is independently selected from those given above, for example trimethylsilyl or trimethylsiloxy.

Groups, which promote the binding to the support (i.e., ionic or ion-forming) should practically be incorporated into the molecule by groups R¹, R², R³ or R⁶. Examples of these structures include the following quaternary ammonium ion-functionalized cyclic (alkyl)(amino)carbene ruthenium complexes:

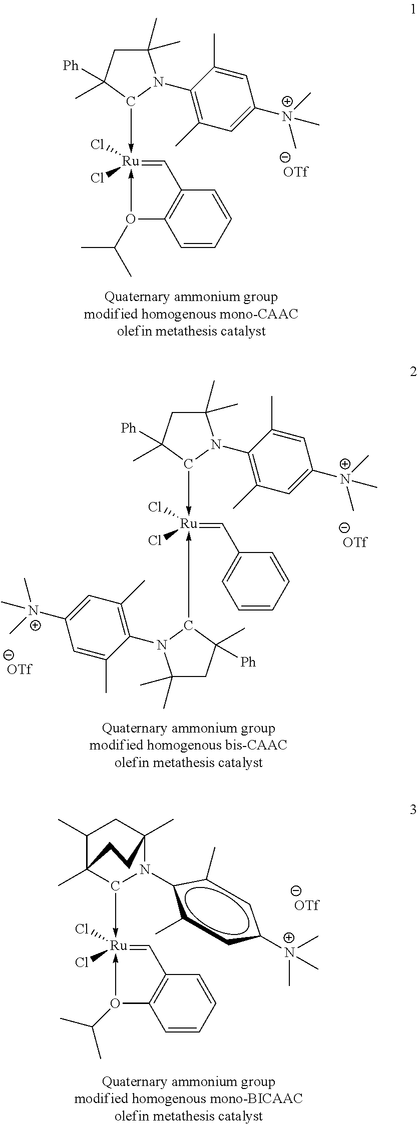

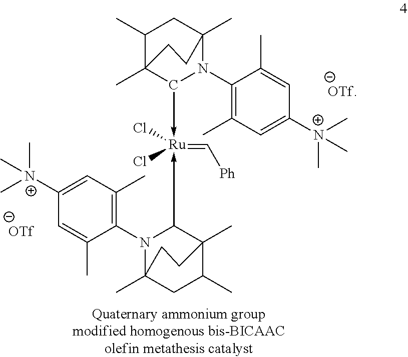

Ionic or ion-forming groups on the CAAC bis-CAAC, BICAAC and bis-BICAAC ligand of the above-mentioned ruthenium complexes have a dual role. On one hand, the complex is stable and active in protic solvents. On the other hand, it has the advantage that the complex can be bound to solids with ionic, ion-exchange or ion-forming properties by ion exchange, ion formation or salt formation, thus yielding a heterogenized homogeneous catalyst. Examples are aluminosilicates or polymer resins with active binding sites such as protons, alkali metal ions, sulfonic acid groups. This allows the complex to be attached to the solid support by ionic interaction (heterogenization). Furthermore, ruthenium complexes containing an ionic or ion-forming group have a significant adsorption affinity for oxide substrates with a high specific surface area, such as γ-aluminum oxide. Therefore, the ruthenium complexes according to the invention can be heterogenized by adsorption.

The invention further relates to ruthenium complex catalysts of formula I, II, III or IV heterogenized as described above.

There are known methods for heterogenizing metathesis catalysts. According to the process described in the application US 2019/0084963, an organosilane coupling agent is used to fix oxo- and imido molybdenum and oxotungstate metathesis catalysts by grafting to the surface of an oxide support covered with hydroxyl groups. The heterogenization of ruthenium complexes is described in the following communication: Keraani et al.: First elaboration of an olefin metathesis catalytic membrane by grafting a Hoveyda-Grubbs precatalyst on zirconia membranes. C. R. Chimie 20 (2017) 952-966. The process involves the attachment of a silylated styrene derivative to the oxide support surface of the ruthenium complex using a coupling agent. According to our invention, the ruthenium complex is more simply attached to solids with ion-exchange or ion-forming properties by complex ion exchange, ion formation or salt formation.

For the isomerization metathesis (ISOMET) reaction, in addition to any homogeneous or heterogeneous metathesis catalyst of our invention, a known heterogeneous-phase solid acid and/or homogeneous-phase isomerization catalyst can be used.

The known heterogeneous catalyst component with double bond isomerization activity at isomerization metathesis reaction is a solid acid catalyst, preferably a zeolite or a polymer functionalized with an acid group. For the known homogeneous phase isomerization catalyst, this is a ruthenium hydride complex, preferably RuH(CO)Cl(PPh₃)₃.

According to a preferred embodiment of the process of our invention, a bifunctional heterogeneous catalyst of our invention is used, which is active in both metathesis and olefin isomerization. The bifunctional catalyst is prepared by heterogenizing the ruthenium complex on a solid acid support.

Due to the relatively low temperature of the ethenolysis reaction, cracking and isomerization of the olefins is unlikely, and therefore the BEA and FAU skeletons with large pores (12-oxygen member ring pore openings) and the MFI, TON and IMF skeletal zeolites with medium pore size (10-oxygen member ring pore openings) may be suitable solid acid supports for the active ruthenium complex. To avoid possible olefin polymerization and rapid catalyst deactivation, zeolite supports with medium pore size and alkali metal form, namely ZSM-5, ZSM-22 and IM-5 zeolites, are preferable. ZSM-22 zeolite is a particularly preferred support.

Suitable polymeric supports for the active complex are cation exchange resins; particularly preferred are modified macro- and microporous styrene copolymers, such as cation exchange styrene-divinylbenzene copolymers, and particularly preferred are polymers containing a sulfonic acid functional group and their alkali metal salts.

### Implementation of the Ethenolysis Reaction:

Using solution-phase, long-chain olefins, the reaction temperature can be between room temperature and the boiling point of the solvent; preferably room temperature. The solvent may be C₅-C₇ alkane, C₇-C₉ aromatic and cycloalkane hydrocarbons or mixtures thereof, which can be easily separable from the product mixture by distillation. Without the use of a solvent, the process can be performed with a paraffin melt at a temperature at which neither the active catalyst component, the ruthenium complex (attached to the support), nor the support structure is damaged.

The reaction can be performed in a batch reactor using a homogeneous catalyst or in a batch reactor or continuous-flow tubular reactor using a heterogenized complex. It is preferable to use the new heterogenized complex in a continuous-flow tubular reactor. The liquid reactant space velocity (LHSV) in the tubular reactor is 0.1-3.0, preferably 0.1-1.0, most preferably 0.1-0.5 h⁻¹. The reaction is carried out at an ethylene pressure of 1-10 bar, preferably 2-3 bar at 2-10 times molar excess ethylene, preferably 7-10 times, most preferably 10 times excess ethylene. The reaction temperature is preferably 80-100° C.

After the dehydrogenation and/or ethenolysis step, the product mixture is separated by gas-liquid-solid separation (FIG. 1).

After the dehydrogenation step, the aim is to separate hydrogen, olefin and paraffin. In order to maintain a hydrogen pressure of 0.5-1.0 bar in the dehydrogenation reactor, part of the hydrogen gas leaving the reactor is recycled back into the reactor. The excess hydrogen can be used as a product outside the alpha-olefin-production technology. The liquid product mixture, optionally containing both paraffin and olefin, is recycled to the ethenolysis operation.

Ethylene and product gases are separated from the ethenolysis product mixture. The ethylene, together with the product gases if applicable, is recycled to the ethenolysis reactor. From the liquid product mixture, fractions with different boiling point ranges are produced by distillation. In the solvent process, the solvent forms the bulk of the most volatile fraction. This is recycled to the solvent of the ethenolysis operation. The highest boiling point fraction is the unconverted long-chain paraffin and olefin. These can be recycled to the dehydrogenation reaction raw material container, from where they can be reintroduced into the dehydrogenation reactor together with the paraffin feedstock. The distillation separation of a fraction rich in terminal olefins is made possible by the fact that the boiling point of alpha-olefins is approximately 10° C. lower than that of internal olefins of the same number of carbon atoms.

The medium-boiling-point alpha-olefin-rich fraction is used as a motor fuel component or as a petrochemical intermediate, for example as a monomer for polymerization, or for alkylation to produce linear alkyl aromatics, such as linear alkylbenzene, depending on technological and market needs. Propylene produced by the ISOMET reaction according to the invention can be used, for example, to produce a biodegradable polymer.

A mixture of liquid olefins prepared according to reaction equation (1) or (2) is suitable for isomerization ethylene metathesis (ethylene ISOMET), regardless of the ratio of terminal to internal olefins. The ISOMET or ethylene ISOMET reaction can occur also in the possible presence of saturated alkanes; it can be carried out either in hydrocarbon solvent or without solvent. An aromatic compound may be present as a co-component or solvent only if the catalyst component of isomerization does not induce alkylation of the aromatic compound.

The catalysts for ISOMET reactions are relatively robust (stable, resistant) ruthenium complexes, or hybrid catalyst systems of ruthenium-aluminum oxide, ruthenium-zeolite and ruthenium-polymer. Catalysts are stable in air, but it is preferable to store them under dry, inert gas. The reaction mixture is prepared in such a way that the reactants, catalyst, equipment and cross-linking agent (ethylene gas) present are dry, free of air and moisture. The quality of the resulting reaction mixture is assessed by the composition of the gas and liquid phases. Gas chromatography and ¹H NMR using an appropriate solvent may be applied to determine the composition. These methods might be applied to the components of the product mixture that can be separated by distillation, too. Volatile components—such as propylene—can be separated from the gas phase with a condenser at an appropriate temperature. For the reactions with ethylene cross-linking agent in a batch reactor, ethylene gas is preferably bubbled through the liquid phase; then recycled to the reactor after the appropriate condensation of volatile products.

A key factor for the quality of the heterogeneous catalyst is that the solid support is prepared prior to the fixing by impregnation of the active component so that it is suitable to catalyze the ISOMET reaction after impregnation. Prior to its use, the oxide support should be activated by heating, and optionally by ion exchange. Preferably, the impregnation solvent has properties that do not modify the solid substrate, i.e., it should be easily removed from the substrate after impregnation, for example by evaporation. Methanol, for example, is an unsuitable solvent for the active ruthenium complex because it binds strongly to the acid sites of the zeolite by coordination. Using absolute dichloromethane solvent for the same impregnation does not present a similar problem.

According to our invention, the catalyst systems and processes used for the preparation of product mixtures containing mainly internal olefins, and then alpha-olefins and/or target product mixtures containing alpha-olefins prepared by ethenolysis of these, will be illustrated by the following examples (solutions), without limiting the invention to examples.

### DESCRIPTION OF THE FIGURES

FIG. 1: Simplified technology process flow diagram without a detailed definition of the catalytic paraffin dehydrogenation and olefin ethenolysis as well as the product mixture separation operations.

FIG. 2: Paraffin composition of Fischer-Tropsch wax.

FIG. 3: ¹H NMR spectrum of a hydrocarbon mixture. [The Hₐ, Hₒ, H_{α}, H_{c1}, H_{c2}, and H_{d} chemical shift ranges belong to different types of hydrogen atoms. Intensities are normalized to the total intensity of the spectrum. Molecular environment of different types of hydrogen atoms: Hₐ—aromatic, Hₒ—olefin, H_{α}—in α position from an aromatic, H_{c1}, H_{c2}—H_{d}—CH₂ and CH₃ protons in various paraffinic molecule parts. (Sun C, Wang Z.: ¹H NMR application in characterizing the refinery products of gasoline, Concepts Magn Reson Part A. 2017; 45A: e21393. https://doi.org/10.1002/cmr.a.21393)].

FIG. 4: GC-MS chromatogram of the product obtained from a paraffin/olefin mixture by ethenolysis (Example 8). The paraffin/olefin mixture was the product of catalytic dehydrogenation of heptadecane (Example 7). The data related to the chromatogram are:

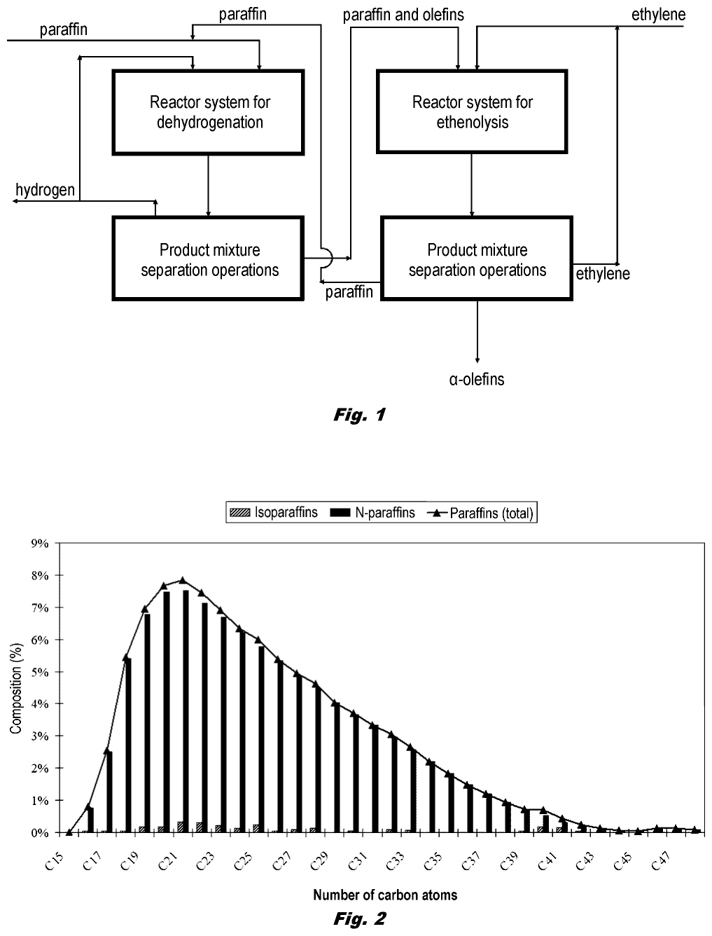

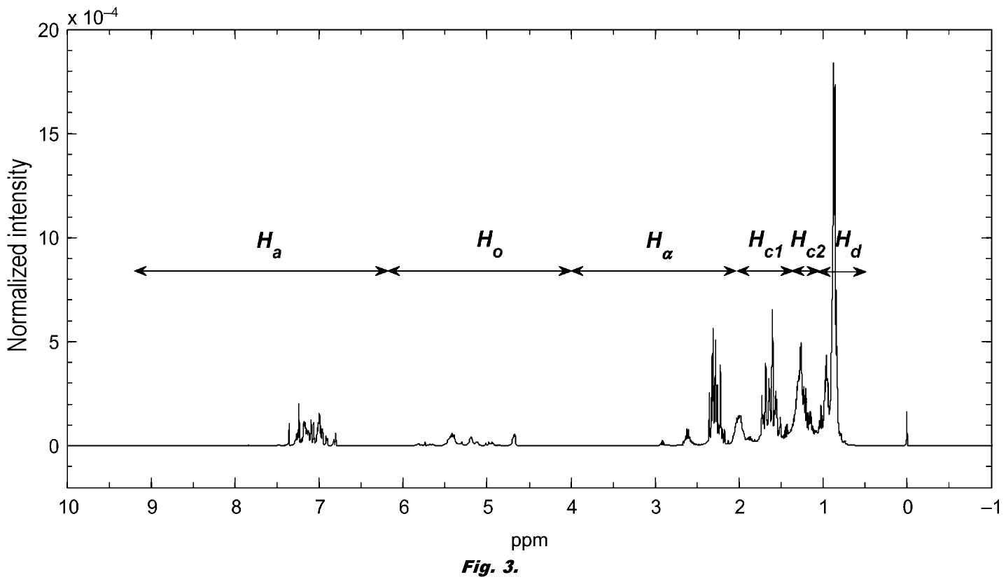

### Total Ion Chromatogram (TIC)

| | Retention | | | | | | | | |
|---|---|---|---|---|---|---|---|---|---|
| Peak# | time | I time | F time | Area | Area % | Height | Height % | A/H | Name |
| 1 | 3.511 | 3.417 | 3.800 | 2674138 | 0.70 | 1037907 | 1.66 | 2.58 | Non-1-ene |
| 2 | 4.319 | 4.108 | 4.650 | 3470313 | 0.91 | 1323792 | 2.12 | 2.62 | Dec-1-ene |
| 3 | 5.082 | 4.833 | 5.508 | 3801610 | 0.99 | 1511970 | 2.42 | 2.51 | Undec-1-ene |
| 4 | 5.787 | 5.633 | 6.033 | 4340724 | 1.14 | 1635124 | 2.61 | 2.65 | Dodec-1-ene |
| 5 | 6.444 | 6.233 | 6.767 | 4254760 | 1.11 | 1756877 | 2.81 | 2.42 | Tridec-1-ene |
| 6 | 7.059 | 6.942 | 7.342 | 4940595 | 1.29 | 2003349 | 3.20 | 2.47 | Tridec-1-ene |
| 7 | 7.634 | 7.492 | 7.650 | 3649215 | 0.95 | 2432780 | 3.89 | 1.50 | Tridec-1-ene |
| 8 | 7.674 | 7.650 | 7.750 | 3153614 | 0.82 | 2450119 | 3.92 | 1.29 | Pentadecane |
| 9 | 8.176 | 8.075 | 8.192 | 6508520 | 1.70 | 3297752 | 5.27 | 1.97 | (E)-Octadec-9-ene |
| 10 | 8.215 | 8.192 | 8.258 | 11735585 | 3.07 | 9014260 | 14.42 | 1.30 | Hexadecane |
| 11 | 8.824 | 8.483 | 8.958 | 307647811 | 80.45 | 27281710 | 43.63 | 11.28 | Heptadecane |
| 12 | 9.035 | 8.958 | 9.125 | 16052322 | 4.20 | 3969268 | 6.35 | 4.04 | Octadec-3-yne |
| 13 | 9.208 | 9.150 | 9.367 | 10201515 | 2.67 | 4818302 | 7.71 | 2.12 | Undecilbenzene |

FIG. 5: The signal of internal hydrogen atoms in vinyl position in the 5.28-5.49 ppm ¹H NMR spectrum range of the alkane/alkene mixture prepared from FT wax by dehydrogenation (Example 9). (The spectrum shows that there is no terminal olefin in the sample.)

FIG. 6: ¹H NMR spectrum of the product obtained from ethenolysis of the mixture obtained by dehydrogenation of FT wax (Example 10). (The signal of the terminal vinyl hydrogen atoms appeared in the range of 4.92-5.10 ppm. The peak at 5.42 ppm chemical shift is associated with the ethylene reactant dissolved in the sample.)

### EXAMPLE 1. PRODUCTION OF A ZEOLITE-SUPPORTED DEHYDROGENATION METAL CATALYST Pt/Na-ZSM-22, Pd/K-ZSM-22, AND Ni/Na-ZSM-22

The ammonium form of the medium pore size ZSM-22 zeolite is used to prepare the catalyst. The zeolite has a specific surface area of 240 m² g⁻¹ and a pore volume of 0.25 cm³ g⁻¹, of which the volume of micropores is 0.08 cm³ g⁻¹, it has a Si/Al ratio of 37 and its framework aluminum content (theoretical ion exchange capacity) is 1.0 mmol g⁻¹. The Ni form is prepared from the ammonium form by ion exchange with Ni acetate solution, while the Pt and Pd forms are prepared via wet impregnation using an aqueous solution of Pt(NH₃)(OH)₂×H₂O, and Pd(NH₃)₄(NO₃)₂, respectively. In impregnation, the air-dried zeolite is contacted with a metal salt solution of the same volume as the pore volume, containing a metal atom (Me, where Me is Pt, Pd, and Ni) in an amount equivalent to the theoretical ion-exchange capacity of the zeolite. The material containing the solution is dried at 110° C. and then calcined at 450° C. for 4 hours in air or treated in a pure oxygen gas stream. The resulting catalyst precursor is reduced in a hydrogen stream at 450° C. The treatment results in the formation of Me/H-ZSM-22 zeolite. It is known that when the metal cations—that are compensating the negative charge of the zeolite lattice—are reduced by hydrogen, the lattice charge compensating role is taken over by protons of acidic character. From the point of view of selective dehydrogenation, the presence of Bronsted acid protons in the Me/H-ZSM-22 catalyst is not advantageous, therefore the powdered Me/HZSM-22 zeolite is rubbed with potassium or sodium nitrate containing an amount of alkali metal equivalent to its cation content and then the salt is decomposed by annealing at 450° C. The resulting Pt/Na and Pd/K and Ni/Na-ZSM-22 catalyst powders are granulated and used in a paraffin dehydrogenation reaction in a continuous-flow tubular reactor. Prior to the reaction, the catalyst is activated by repeated hydrogen reduction, preferably in situ, in the tubular reactor used for paraffin dehydrogenation.

### EXAMPLE 2. PREPARATION OF BICAAC LIGAND

The cyclic iminium precursor of the BICAAC ligand of the ruthenium-containing catalyst complex for the ISOMET process can be prepared according to equation (5).

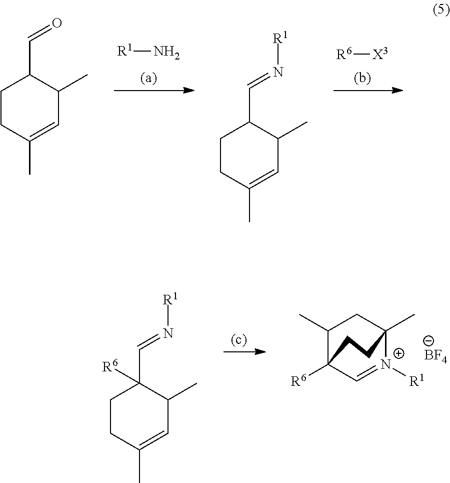

### EXAMPLE 2.1: FIRST STEP IN THE PREPARATION OF BICAAC LIGAND (A)

The R¹—NH₂ primer amine is dissolved in anhydrous dichloromethane (concentration of aniline is approximately 1 mol/dm³), 1.0 molar equivalent of 2,4-dimethylcyclohex-3-ene-1-carbaldehyde is added, and then molecular sieve—dried in prior under vacuum at 200° C.—is added to the mixture (in an amount of at least 1 g/mmol substrate aldehyde). The molecular sieve must have the property to bind the water produced in the reaction, preferably a zeolite type with 3 Å pore size. Then, the resulted mixture is allowed to stand at room temperature for 16 hours without stirring, instead, shaking gently a few times. The molecular sieve is filtered out of the mixture, the solution is evaporated, and the product (imine) is obtained. The purity of the product is determined by NMR and gas chromatography. The product is stored in an anhydrous environment, protected from light, below 10° C. The reaction can also be performed in another organic solvent that dissolves both starting materials (e.g., toluene).

Example amounts for the reaction of 2,6-dimethyl-4-dimethylaminoaniline and 2,4-dimethylcyclohex-3-ene-1-carbaldehyde:
2,6-dimethyl-4-dimethylaminoaniline 33.83 mmol, that is 5.54 g
2,4-dimethylcyclohex-3-ene-1-carbaldehyde: 33.83 mmol, that is 5.00 ml
Molecular sieve with a pore size of 3 Å: 20.0 g
Dichloromethane solvent: 40.0 ml
The weight of the obtained product (N-(2,6-dimethyl-4-dimethylaminophenyl)-1-(2,4-dimethylcyclohex-3-en-1-yl) methanimine) is 9.40 g and the reaction yield is 98%.

Representative analytical data for the compound prepared according to the above example.

Since two isomers of the 2,4-dimethylcyclohex-3-ene-1-carbaldehyde forms in the reaction (positions 1 and 2 of the cyclohexene ring can be cis and trans relative to each other), some signals are duplicated. The chemical shifts of both isomers can be seen below. For this molecule, the ratio of the two isomers is 28:72. The unassigned CH and CH₂ signals of the cyclohexene ring are marked with an asterisk.

¹H NMR (300 MHz, CDCl₃) δ: 7.68 (d, J=5.9 Hz, 0.28×1H, CH═N, minor isomer), 7.56 (d, J=5.9 Hz, 0.72×1H, CH═N, minor isomer), 6.48 (s, 2H, CH_{Ar}), 5.35 (s, 0.28×1H, C═CH minor isomer), 5.28 (s, 0.72×1H, C═CH, major isomer), 2.89 (s, 6H, NMe₂), 2.80-2.17 (m, 3H*), 2.11 (s, 6H, ArMe₂), 2.06-1.91 (m, 3H*), 1.70 (s, 0.72×3H, C-Me, major isomer), 1.67 (s, 0.28×3H, C-Me, minor isomer), 1.11 (d, J=6.9 Hz, 0.72×3H, CH-Me major isomer), 1.03 (d, J=7.2 Hz, 0.28×3H, CH-Me, minor isomer).

¹³C NMR (75 MHz, CDCl₃) both isomers δ: 171.83, 171.24, 150.16, 147.40, 133.30, 127.91, 126.52, 113.27, 48.11, 44.30, 41.38, 32.83, 32.21, 29.19, 28.44, 26.24, 24.21, 23.66, 20.78, 18.97, 18.21.

HRMS calculated m/z: 285.232525, measured: 285.2323 ([M+H]⁺ C₁₉H₂₈N₂H).

### EXAMPLE 2.2: SECOND STEP OF THE PREPARATION OF BICAAC LIGAND (B)

The imine obtained in Example 2.1 is further converted in an alkylation reaction. The imine is dissolved in anhydrous THF solvent at inert atmosphere, cooled to 0° C., and at least 1.0, preferably 2.0 molar equivalents of lithium diisopropylamide—which may be in the form of a commercially available solution in ether or hydrocarbon solvent—is added. After half an hour, the mixture is allowed to warm to room temperature and stirred for four hours in an inert medium. The reaction is then cooled again to 0° C. and an R⁶—X³ alkylating agent is added to give the substituent R⁶ in equation (5). The leaving group X³ is optionally chlorine or a heavier halogen, preferably iodide, possibly a derivative of a strong organic carboxylic acid or sulfonic acid (e.g., tosylate or triflate). After adding the alkylating agent, the mixture is stirred at room temperature for 16 hours in a sealed flask. As a processing step, water and hexane are carefully added. The organic phase is washed in a separatory funnel with sodium bisulfite solution, then with water. After separation, the desired alkylated imine product is obtained by evaporation. The purity of the product is determined by NMR and gas chromatography. The product is stored in an anhydrous environment, protected from light, below 10° C. The reaction can be carried out in other ether-type solvents (e.g., diethyl ether) or hydrocarbon solvents (e.g., hexane). A requirement for the solvent to be anhydrous and not to react with reagents or substrate.

Example quantities for the alkylation of imine (N-(2,6-dimethyl-4-dimethylaminophenyl)-1-(2,4-dimethylcyclohex-3-en-1-yl)methanimine) with methyl iodide formed in the reaction of Example 2.1
N-(2,6-dimethyl-4-dimethylaminophenyl)-1-(2,4-dimethylcyclohex-3-en-1-yl)methanimine: 30.00 mmol, 8.08 g
LDA solution (in 2 M THF-hydrocarbon solvent): 60.00 mmol, 30.0 ml
THF solvent: 40.0 ml
Methyl iodide: 60.00 mmol, 3.74 ml

The weight of the obtained product (N-(2,6-dimethyl-4-dimethylaminophenyl)-1-(1,2,4-trimethylcyclohex-3-en-1-yl)methanimine) is 8.60 g and the reaction yield is 96%.

Representative analytical data for the compound prepared according to the above example.

¹H NMR (300 MHz, CDCl₃) δ: 7.69 (s, 1H, CH═N), 6.48 (s, 2H CH_{Ar}), 5.24 (s, 1H, olefinic C═CH), 2.89 (s, 6H, Nme₂), 2.73-2.59 (m, 1H*), 2.32-2.11 (m, 2H*), 2.08 (s, 6H, Ar2.6-Me₂), 2.06-1.93 (m, 2H*), 1.80-1.70 (m, 2H*), 1.66 (s, 3H, C-Me), 1.27 (s, 3H, C-Me), 1.02 (d, J=7.3 Hz, 3H, CH-Me).

¹³C NMR (75 MHz, CDCl₃) δ: 173.37, 147.29, 142.79, 133.29, 127.72, 126.26, 113.33, 113.27, 41.57, 41.44, 39.01, 32.79, 28.08, 23.68, 23.36, 18.93, 17.28.

HRMS: calculated m/z: 299.248175, measured: 299.2479 ([M+H]⁺ C₂₀H₃₀N₂⁺).

### EXAMPLE 2.3: THIRD STEP OF THE PREPARATION OF BICAAC LIGAND (C)

The alkylated imine obtained by the process of Example 2.2 is dissolved in anhydrous 1,4-dioxane (hereinafter referred to as dioxane) and added under inert conditions to an also anhydrous dioxane solution containing 5 molar equivalents of hydrogen chloride in a concentration of at least 3 mol dm⁻³. This solution may also be prepared by dissolving anhydrous acetyl chloride in anhydrous dioxane and adding equimolar amount of dry methanol at 0° C. If the molecule to be reacted also contains a group that binds the hydrogen chloride (e.g., amine or another protecting group), the hydrogen chloride should be used in excess of this group as well. The resulting mixture is sealed, stirred, and heated for 48 hours at 80° C. This requires preferably a pressure vessel having a thick wall. At the end of the reaction, the mixture is allowed to cool to room temperature and the sodium bicarbonate solution is added to decompose the hydrogen chloride until the gas evolution has ceased. In addition, further amount of dichloromethane—the volume of which is equal to at least half of the reaction volume—is added to the mixture. The resulting material is separated in a separatory funnel and the organic phase is concentrated by evaporation. The evaporation residue is dissolved in dichloromethane to form a concentrated solution, and two molar equivalents of ammonium tetrafluoroborate are added in the form of its saturated aqueous solution. The mixture is stirred at room temperature for two hours and then subjected to phase separation. The organic phase is dewatered with a drying agent (e.g., sodium sulfate) and then evaporated. Diethyl ether, hexane or other alkane, cycloalkane is added to the resulting crude product, in an amount at least seven times its weight, and the product is recrystallized. The quality of the crystallized product is determined by NMR (¹H, ¹³C, and ¹⁹F) and, if necessary, it is further recrystallized from hydrocarbon solvent. The product may be stored at room temperature in air, but must be dried under a strong vacuum (max. 1 mbar for at least 6 hours) before complexation and further manipulations must be carried out in a water- and oxygen-free environment. The reaction may be carried out with other aprotic solvents, in which the hydrogen chloride is soluble at a concentration of at least 1 mol/dm³ and does not react with alkylated imine prepared according to Example 2.2 (e.g., diethyl ether). The reaction may be performed with other strong acids (e.g., hydrogen bromide) instead of hydrogen chloride, if it does not lead to a side reaction on the rest of the molecule. The ammonium tetrafluoroborate anion exchange can also be performed with other ionic compounds, in which the anion does not (or only to a very limited extent) exhibit coordinating properties (e.g., perchlorate, PF₆⁻, BARF ions, etc.). The anion exchange can be omitted only if the anion of the ring-closed product is identical to the X¹ and X² groups of the future ruthenium complex.

Example amounts of the cyclization of the alkylated imine (N-(2,6-dimethyl-4-dimethylaminophenyl)-1-(1,2,4-trimethylcyclohex-3-en-1-yl)methanimine)
N-(2,6-dimethyl-4-dimethylaminophenyl)-1-(1,2,4-trimethylcyclohex-3-en-1-yl)methanimine: 25.00 mmol, 7.46 g
Hydrogen chloride solution (in 3 M dioxane solvent): 250.00 mmol, 83.33 ml
Dioxane solvent: 15.0 ml
Ammonium tetrafluoroborate: 5.24 g

The weight of the resulting (ring-closed) product, 2-(2,6-dimethyl-4-dimethylaminophenyl)-1,4,5-trimethyl-2-azabicyclo[2.2.2]oct-2-en-2-ium tetrafluoroborate, is 1.35 g and the reaction yield is 14%.

Representative analytical data for the compound prepared according to the above example.

¹H NMR (500 MHz, CDCl₃) δ: 9.06 (s, 1H, CH═N⁺), 6.37 (s, 2H, Ar_{CH}), 2.96 (s, 6H, NMe₂), 2.44 (dd, J=14.0, 10.1 Hz, 1H*), 2.30-2.21 (m, 1H*), 2.20 (s, 3H, Ar-Me), 2.14 (s, 3H, Ar-Me), 2.12-2.01 (m, 2H*), 2.00-1.91 (m, 1H*), 1.89-1.81 (m, 1H*), 1.63 (s, 3H, C-Me), 1.62-1.56 (m, 1H*), 1.24 (s, 3H, C-Aft), 1.07 (d, J=7.2 Hz, 3H, CH-Me).

¹³C NMR (75 MHz, CDCl₃) δ: 193.76, 150.85, 133.57, 132.88, 127.92, 112.03, 111.95, 69.37, 44.92, 43.82, 40.13, 38.46, 33.32, 33.17, 21.42, 20.61, 20.56, 19.21, 18.80.

¹⁹F NMR (282 MHz, CDCl₃) δ: −152.32 (¹¹BF₄⁻), −152.37 (¹⁰BF₄⁻.

HRMS: calculated m z: 299.248175, measured: 299.2481 (with a C₂₀H₃₁N₂⁺ molecular formula of the organic cation).

### EXAMPLE 3. PREPARATION OF BICAAC COMPLEXES

These reactions must be performed under anhydrous conditions, therefore the solvents have to be dried; the glassware should be treated by heating and vacuuming before use; as well as the substrate and reagents have to be anhydrous and oxygen-free. Preferably, the work is performed in a laboratory glovebox filled with dry, inert gas (nitrogen, argon), where the water and oxygen concentrations are less than 10 ppm in the atmosphere. The cyclic iminium compound prepared according to example 2.3 can be incorporated into a ruthenium complex [reaction (6) or (7)] catalyzing the metathesis of olefins as follows.

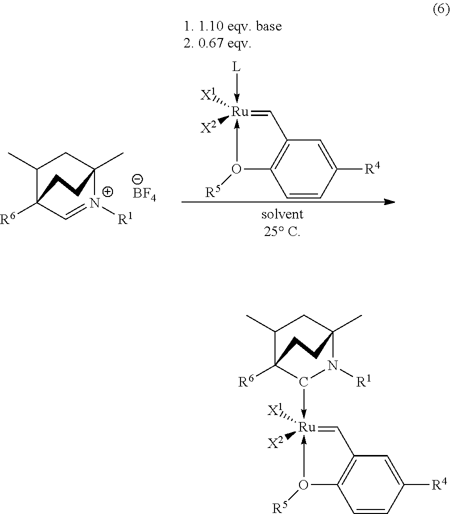

### EXAMPLE 3.1: PREPARATION OF A MONO-BICAAC COMPLEX

The cyclic iminium compound, prepared as in Example 2.3, is weighed into a small sample bottle and suspended in an ether- or hydrocarbon-type solvent. Then a base—with a pKₐ of at least 32 for its conjugated acid form (preferably alkali hexamethyldisilyl azide compounds, even in solution)—is added and the contents of the container are stirred until the iminium compound dissolves, indicating the formation of free carbene from the precursor salt. Thereafter, the ruthenium(II) compound according to equation (6) is added in its concentrated solution—in the same solvent as used for the suspension of the iminium compound—containing the alkoxy benzylidene ligand, and L ligands with reduced binding to the metal compared to the carbene (ideally a tertiary amine or a phosphine such as pyridine or tricyclohexylphosphine). The ligand exchange is completed in three hours during stirring, and the reaction can be monitored by NMR measurements and sometimes by naked eye due to the colour change that occurs. The mono-BICAAC complex can be recovered from the resulting mixture by chromatography or recrystallization. The solvents used in the reaction can include diethyl ether, tetrahydrofuran, dioxane, hexane, cyclohexane, toluene, benzene, xylene.

Example amounts for the complexation of the iminium salt (2-(2,6-dimethyl-4-dimethylaminophenyl)-1,4,5-trimethyl-2-azabicyclo[2.2.2]oct-2-en-2-ium tetrafluoroborate) complexed with the Hoveyda-Grubbs 1st generation complex (CAS: 203714-71-0, in this case L=tricyclohexylphosphine).
Hoveyda-Grubbs 1st generation complex: 167 μmol, 100.0 mg
2-(2,6-dimethyl-4-dimethylaminophenyl)-1,4,5-trimethyl-2-azabicyclo[2.2.2]oct-2-en-2-ium tetrafluoroborate: 250 μmol, 96.5 mg
lithium hexamethyldisilazide (1 M THF solution): 275 μmol, 275 μl
THF solvent: 10.0 ml

The weight of the resulting complex, {2-[2,6-dimethyl-4-dimethylaminophenyl]-1,4,5-trimethyl-2-azabicyclo[2.2.2]octane-3-ylidene}{2-isopropoxybenzylidene}ruthenium(II) dichloride is 64.9 mg and the reaction yield is 63%.

Representative analytical data for the compound prepared according to the above example.

¹H NMR (500 MHz, CDCl₃) δ: 16.47 (s, 1H, Ru═CH), 7.54 (td, J=8.4, 1.7 Hz, 1H, CH_{Bzy}), 7.00-6.85 (m, 3H, CH_{Bzy}), 6.59 (dd, J=13.6, 2.7 Hz, 2H, CH_{Ar}), 5.16 (sept, J=6.1 Hz, 1H, CH_{iPrO}), 3.06 (s, 6H, NMe₂), 2.82 (s, 3H, C-Me_{BIAAC}), 2.43 (dd, J=11.2, 5.1 Hz, 1H, CH_{2BICAAC}), 2.27 (s, 3H, Me-Ar), 2.17 (s, 3H, Me-Ar), 2.12 (dd, J=13.0, 10.5 Hz, 1H, CH_{BICAAC}), 2.04-1.90 (m, 2H, CH_{BICAAC} and 1H of CH_{2BICAAC}), 1.75 (d, J=6.3 Hz, 3H, Me_{iPrO}), 1.74 (d, J=6.3 Hz, 3H, Me_{iPrO}), 1.73-1.64 (m, 2H, 1H of CH_{BICAAC} and 1H of CH_{BICAAC}), 1.56-1.48 (m, 1H, CH_{BICAAC}), 1.32 (d, J=7.1 Hz, 3H, CH-Me_{BICAAC}), 1.10 (s, 3H, C-Me_{BICAAC}).

¹³C NMR (126 MHz, CDCl₃) δ: 306.40 (Ru═CH), 263.55 (BICAAC carbene), 151.92, 150.28, 144.99, 137.67, 137.63, 135.81, 130.81, 124.12, 122.10, 113.12, 113.11, 112.97, 74.70, 66.46, 54.07, 46.18, 40.80, 40.01, 33.90, 30.42, 23.96, 22.24, 22.14, 21.40, 21.28, 20.55, 19.80.

HRMS: calculated m z: 583.2029, measured: 583.2033 ([M−Cl]⁺: C₃₀H₄₂n₂OClRu⁺).

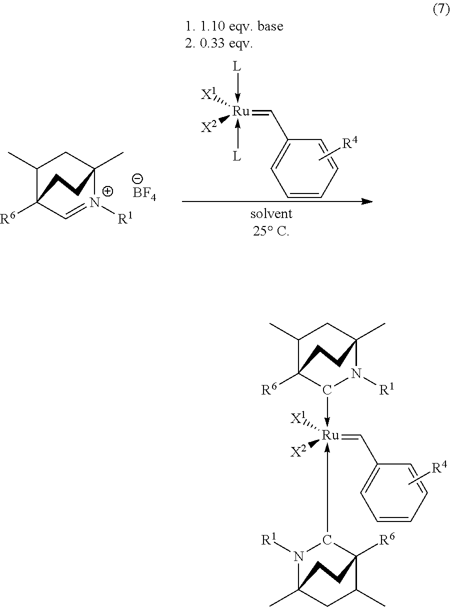

### EXAMPLE 3.2: PREPARATION OF A BIS-BICAAC COMPLEX

The cyclic iminium compound, prepared as in Example 2.3, is weighed into a small sample bottle and suspended in an ether- or hydrocarbon-type solvent. Then a base—with a pKₐ of at least 32 for its conjugated acid form (preferably alkali hexamethyldisilyl azide compounds, even in solution)—is added and the contents of the container are stirred until the iminium compound dissolves, indicating the formation of free carbene from the salt. Thereafter, the ruthenium(II) compound according to equation (7) is added in its concentrated solution—in the same solvent as used for the suspension of the iminium compound—containing the optionally substituted benzylidene ligand, and two L ligands with reduced binding to the metal compared to the carbene (ideally a tertiary amine or a phosphine such as pyridine or tricyclohexylphosphine). The ligand exchange is performed in three hours during stirring, and the reaction can be monitored by NMR measurements and sometimes by naked eye due to the colour change that occurs. The mono-BICAAC complex can be recovered from the resulting mixture by chromatography or recrystallization. The solvents used in the reaction can include diethyl ether, tetrahydrofuran, dioxane, hexane, cyclohexane, toluene, benzene, xylene.

Example amounts for the complexation of the iminium salt (2-(2,6-dimethyl-4-dimethylaminophenyl)-1,4,5-trimethyl-2-azabicyclo[2.2.2]oct-2-en-2-ium tetrafluoroborate) complexed with the 1st generation Grubbs complex (CAS: 172222-30-9, in this case L=tricyclohexylphosphine).
Grubbs 1st generation complex: 122 μmol, 100.0 mg
2-(2,6-dimethyl-4-dimethylaminophenyl)-1,4,5-trimethyl-2-azabicyclo[2.2.2]oct-2-en-2-ium tetrafluoroborate: 365 μmol, 141.0 mg
lithium hexamethyldisilazide (1 M THF solution): 402 μmol, 402 μl
THF solvent: 10.0 ml

The weight of the resulting complex {2-[2,6-dimethyl-4-dimethylaminophenyl]-1,4,5-trimethyl-2-azabicyclo[2.2.2]octane-3-ylidene}{benzylidene}ruthenium(II) dichloride is 40.6 mg and the reaction yield is 39%.

HRMS: calculated m/z: 823.4024, measured: 823.4024 ([M−Cl]⁺ C₄₇H₆₆ClN₄Ru⁺).

The purity of the product can also be verified by NMR measurement in addition to HRMS measurement. The exact structure cannot be determined by one-dimensional measurements because many diastereomers are formed in the mixture. A product is considered pure if
in ¹H NMR
   no characteristic singlet of the protonated ligand is visible between 12.0 and 8.0 ppm,
   the benzylidene position Ru═CH hydrogen sign (singlet) is detectable between 25.0 and 15.0 ppm;
in the ¹³C NMR spectrum, above 250.0 ppm the presence of carbon atoms is seen at the benzylidene position at Ru═CH and the carbene type carbon atom of the BICAAC ligand;
no signal is visible in the ¹⁹F and ³¹P NMR spectra.

EXAMPLE 4. PREPARATION OF MONOCYCLIC (CAAC, BIS-CAAC) AND BICYCLIC (BICAAC, BIS-BICAAC) ALKYLAMINO-CARBENE COMPLEXES CONTAINING A QUATERNARY AMMONIUM ION GROUP AND THEIR HETEROGENIZATION ON ZEOLITE SUPPORTS

a) A Ru(II) complex containing a benzylidene, substituted benzylidene, indenylidene, or substituted indenylidene ligand [ideally Hoveyda-Grubbs (HG1) or Grubbs (G1) complexes] is reacted with a monocyclic (CAAC), and bicyclic (BICAAC) alkylamino-carboxylic ligands, respectively, which carbenes contain an ionic or ion-forming functional group, preferably a tertiary amine and a quaternary ammonium functional group. The structural formulae of the prepared quaternary ammonium group-containing monocyclic and bicyclic (alkyl)(amino)carbene ruthenium complexes is that of compounds 1, 2, 3, 4 above. TfO⁻ represents triflate (i.e. trifluoromethanesulfonate) ion. To prepare the catalysts, the precursor of the carbene ligand is first synthesized in five process steps. The resulting precursor is then reacted with HG1 or G1 ruthenium complex. The preparation procedure of the complex containing the ligand functionalized with an ammonium group is described in detail in the following communication: M. Nagyhizi and co-workers: ChemCatChem 12, (2021) 1953-1957. The BICAAC complexes were prepared according to the former principle using the BICAAC ligand prepared as in Example 3. The preparation of stable, bicyclic (alkyl)(amino)carbenes was first described in: E. Tomas-Mendivil and co-workers: J. Am. Chem. Soc. (2017), 139, 7753-7756.

b) A solution of bis-CAAC and BICAAC metathesis catalyst saturated with dichloromethane at a concentration of approx. 20-30 mM (mmol) is prepared. For the heterogenization of the ruthenium complex, either K-ZSM-22 zeolite with a particle size of 300-425 μm, as described in Example 1, or commercially available Na—Y zeolite (Si/Al=2.5) is used. The solid support is dried by heating in vacuum (>100° C.) before impregnation. The solution of the ruthenium complex is added to the zeolite and stirred for 12 hours at room temperature in an inert atmosphere. At the end of the reaction, the suspension is filtered, washed several times with dichloromethane and then the resulted greenish solid material is dried in vacuum. The impregnation is sufficiently effective if the impregnating solution used contains more active complexes than the complex adsorption capacity of the support. This can be easily checked with the naked eye or by ultraviolet-visible (UV-vis) spectroscopy because the metathesis catalyst is colored. The solvent must be colored even after the impregnation is complete. When the adsorption equilibrium is reached, the impregnated catalyst is filtered off and washed with the solvent used to dissolve the catalyst until the filtrate no longer contains any solute. The amount of active component impregnated on the solid support can be determined by mass measurement of the residue remained after evaporation of the mother liquor.

The amount of ruthenium bound on zeolite from both complexes is approximately 0.12 mmol_{cat} g_{zeolite}⁻¹. The zeolite supported heterogenized complex catalyst is tested in olefin ethenolysis.

EXAMPLE 5. PREPARATION OF MONOCYCLIC (CAAC, BIS-CAAC) AND BICYCLIC (ALKYL)(AMINO)CARBENE (BICAAC) RUTHENIUM COMPLEXES CONTAINING A QUATERNARY AMMONIUM ION GROUP FOR ETHENOLYSIS REACTION BOUND TO A POLYMERIC SUPPORT

The procedure is similar to the one described in Example 4. To heterogenize the ruthenium complex, the hydrogen form of Amberlyst 15 is used, that is a styrene-divinylbenzene-matrix resin functionalized with sulfonic acid groups, which has a particle size of 300-425 μm, a specific surface area of 53 m² g⁻¹, an average pore size of 30 nm, and a theoretical ion exchange capacity of ˜4.7 meq·g⁻¹. The CAAC or BICAAC ruthenium complex prepared according to Example 4 is used to prepare a solution of 10 mM (mmol) using dichloromethane solvent. The Amberlyst 15 resin is pretreated by evacuation (below 1 mbar) and by heat treatment (200° C.) for 6 hours. To the solution of the ruthenium complex pre-treated Amberlyst 15 resin is added in an amount to make the mass of the resin ten times greater than that of the ruthenium complex dissolved in dichloromethane. The resulting mixture first is stirred under a dry inert gas atmosphere (e.g., nitrogen or argon) for 12 hours, then the solution is separated from the resin by decantation and finally the resin is washed with pure dichloromethane until the liquid removed no longer contains the complex to be impregnated. The surface coverage of the resin is 0.10 mmol_{cat}/g_{Amberlyst}.

EXAMPLE 6. PREPARATION OF MONOCYCLIC (CAAC, BIS-CAAC) AND BICYCLIC (ALKYL)(AMINO)CARBENE (BICAAC) RUTHENIUM COMPLEXES CONTAINING QUATERNARY AMMONIUM ION GROUP FOR ETHENOLYSIS REACTION BOUND TO ALUMINUM OXIDE SUPPORT

The procedure is similar to the one described in Example 4. For the heterogenization of ruthenium complex, aluminum oxide is used, which is pretreated by evacuation (below 1 mbar) and heating (200° C.) for 6 hours. The aluminum oxide used is preferably of Brockmann I reactivity, 40-300 μm particle size, 60 Å pore size, used for organic preparative chromatography. The CAAC or BICAAC ruthenium complex prepared according to Example 4 is used to prepare a solution of 10 mM (mmol) using dichloromethane solvent. To the solution of the ruthenium complex pretreated aluminum oxide is added in an amount to give a mass ten times greater than that of the ruthenium complex dissolved in dichloromethane. The resulting mixture is stirred under a dry, inert gas atmosphere (e.g., nitrogen or argon) for 12 hours, the solution is separated from the solid phase by decantation, and the impregnated aluminum oxide is washed with pure dichloromethane until the liquid leaving no longer contains the complex to be impregnated. The surface coverage of the support is 0.12 mmol_{cat}/gₛᵤₚₚₒᵣₜ.

### EXAMPLE 7. CATALYTIC DEHYDROGENATION OF HEXADECANE AND HEPTADECANE ON A ZEOLITE-SUPPORTED METAL CATALYST PREPARED ACCORDING TO EXAMPLE 1

Hexadecane and heptadecane are dehydrogenated in a continuous-flow tubular reactor. Hexadecane has a melting point of 18° C. and a boiling point of 287° C. Heptadecane has a melting point of 21-23° C. and a boiling point of 302° C. A Teledyne ISCO 100DM Syringe Pump, a high-pressure solvent delivery pump is used to feed the liquid reactant into the reactor. Hydrogen is added to the reactant via a mass flow controller. To perform catalytic measurements, 250 mg of 5% Pt/Na-ZSM-22 catalyst is diluted to 5 cm³ with inert silicon carbide and loaded into a 0.5 cm diameter reactor tube. Before starting the reaction, the catalyst is activated under atmospheric pressure at 480° C. for 3 hours in a pure hydrogen stream under reaction conditions.

The product mixture is cooled to room temperature and the products—which are liquid at room temperature—are separated from the gaseous product mixture. The product mixture is drained into a sample vessel every hour; the first sample after the reaction is started is not analyzed, only the subsequent ones (equilibrium is then considered to have been reached). Gas chromatograph-mass spectrometry (GC-MS) equipment with a Zebron ZB-WAXplus column (L 60.0 m×ID 0.32 mm×df 0.5 μm) is used to identify the components of the liquid sample. The conditions of the analysis: He carrier gas and (50° C., 5 min; 248° C. (10° C./min), 20 min) temperature program. The gas space is also sampled hourly and analyzed with ShinCarbon ST (L 2.0 m×ID ⅛ in.×OD 2.0 mm) column and a gas chromatograph equipped with FID and TCD detectors. The conditions of the analysis: Ar carrier gas and (100° C., 3 min; 250° C. (12° C./min), 16 min) temperature program.

The conditions and results of the dehydrogenation reaction are summarized in Table 1.

TABLE 1
Hexadecane dehydrogenation on 5% Pt/Na-ZSM-22 catalyst

| | | | | |
|---|---|---|---|---|
| Temperature, ° C. | 450 | 450 | 480 | 480 |
| Pressure, bar | 1.5 | 1.5 | 1.5 | 1.5 |
| Hexadecane loading/feed, g h⁻¹ | 5.0 | 5.0 | 5.0 | 5.0 |
| LHSV*, h⁻¹ | 20 | 20 | 20 | 20 |
| H₂ loading/feed, ml/min | 50 | 50 | 50 | 50 |
| GHSV**, h⁻¹ | 12,000 | 12,000 | 12,000 | 12,000 |
| Sample collection | 2 hours | 3 hours | 2 hours | 3 hours |
| Weight of liquid product, g | 4.95 | 4.67 | 4.87 | 4.74 |
| Hexadecane, wt % | 87.6 | 92.6 | 86.0 | 84.5 |
| Hexadecene, wt % | 12.4 | 7.4 | 14.0 | 15.5 |

*LHSV: liquid hourly space velocity
**GHSV: gas hourly space velocity

No product other than 1-hexadecene was produced under the experimental conditions used. About 10-15% of hexadecane is converted to C₁₆ olefins. No diolefins or C₁₆ skeletal isomers were formed.

Heptadecane was also dehydrogenated on the 5% Pt/Na-ZSM-22 catalyst. Using the experimental conditions presented in Table 1, similar olefin yields were obtained as in case of hexadecane. The 5% Pd/Na-ZSM-22 catalyst showed a high monoolefin selectivity, similar to that of the Pt catalyst, but its activity was only about 1/10 that of the corresponding Pt catalyst. When Ni zeolite catalysts was used, 1-5 wt % of gaseous alkane—mainly methane—appeared in the product mixture. This has reduced the olefin yield.

The distribution of monoolefin double bond isomers was deduced from the results of ethenolysis experiments performed with the liquid product.

### EXAMPLE 8. ETHENOLYSIS OF A PARAFFIN-OLEFIN MIXTURE OBTAINED BY DEHYDROGENATION OF HEPTADECANE

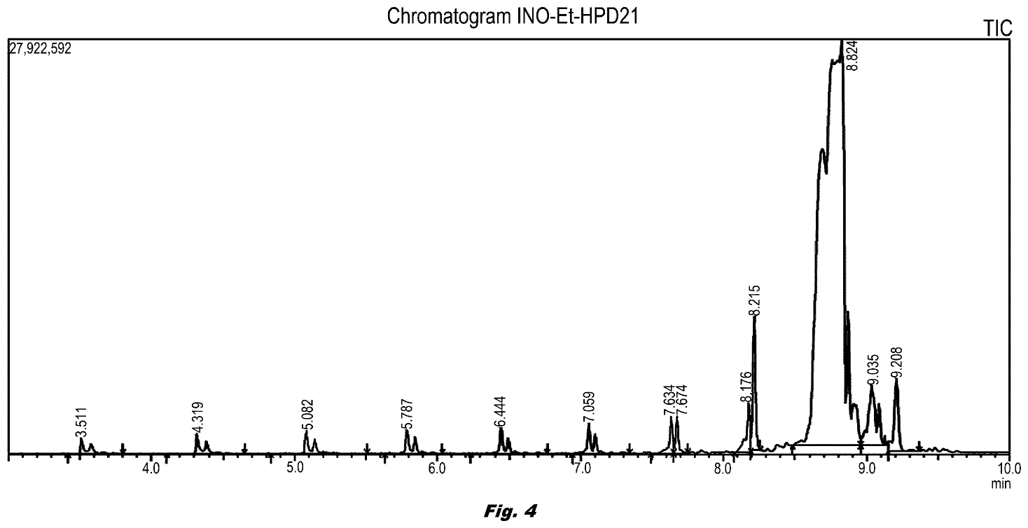

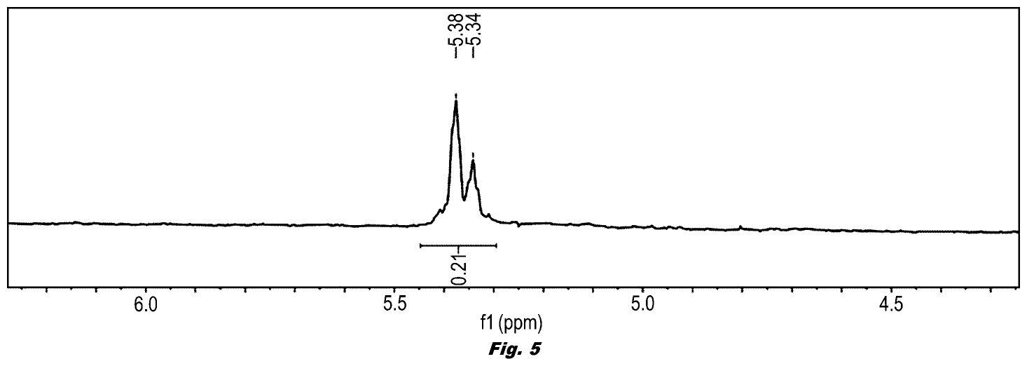

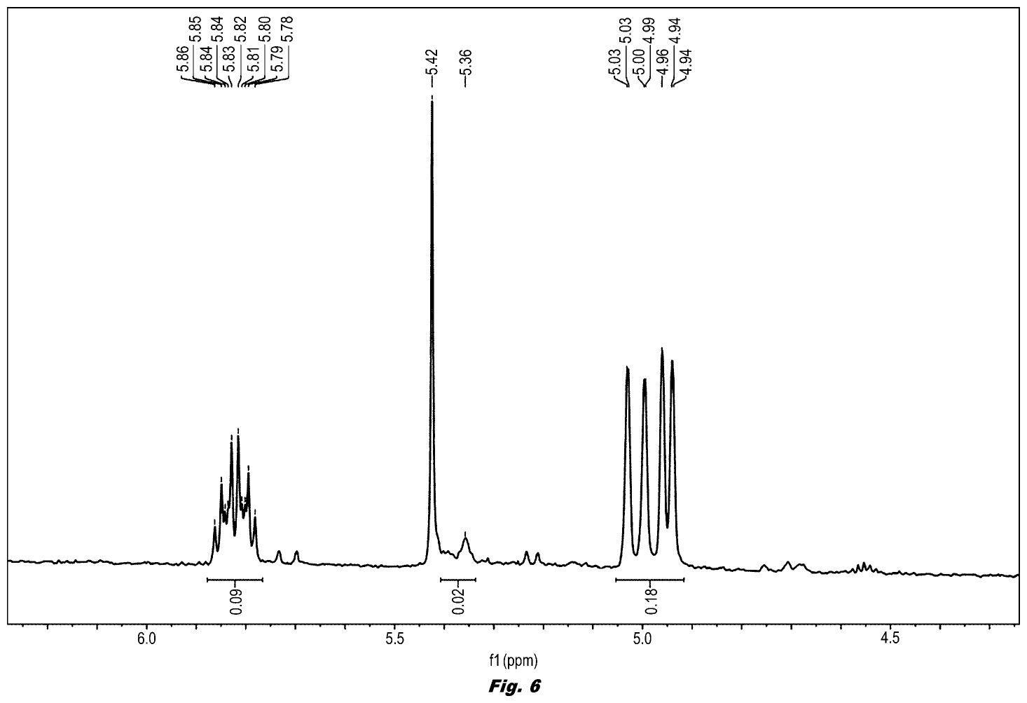

The ethenolysis of the product mixture—that is produced from the dehydrogenated heptadecane of Example 7—is performed on a BICAAC ruthenium complex catalyst (prepared according to Example 3), which is immobilized on a Na—Y zeolite catalyst of Example 4(b). The reactants are weighed under an inert atmosphere in a laboratory glovebox, ideally filled with an inert gas. 2 cm³ of the liquid product of heptadecane dehydrogenation and 7 mg catalyst are weighed into a pressure vessel and then the mixture is placed under 10 bar ethylene pressure. The reaction mixture is stirred at 75° C. for 4 hours. The liquid is separated from the catalyst and its composition is determined by GC-MS analysis. The gas chromatogram recorded is shown in FIG. 4. The highest intensity chromatographic peak belongs to the unconverted heptadecane. 1-heptadecene (C₁₇⁼) is present in very low concentrations in the product mixture, but the full spectrum of C₃⁼-C₁₆⁼ alpha olefins is present, indicating that the ethenolysis reaction has converted the total C₁₇⁼ olefin content of the reaction mixture to C₃⁼-C₁₆⁼ alpha olefins.

### EXAMPLE 9. DEHYDROGENATION OF FISCHER-TROPSCH (FT) WAX

Our example illustrates the production of alpha-olefins according to our invention from a bioparaffin mixture, namely from FT wax. The carbon source of the syngas—used for the low-temperature FT reaction, leading to the production of FT wax—was a raw material of biological origin. Paraffin has a composition essentially indistinguishable from that obtained from fossil carbon sources, but is a preferred feedstock for our process due to its lower contaminant content. Its properties are given in Table 2 and the paraffin composition is shown in FIG. 2.

TABLE 2
Main properties of the FT wax

| Property | Value |
|---|---|
| Pour point, ° C. | 72 |
| Density, g cm⁻³ (solid) | 0.89 |
| n-paraffin content, % | 97.4 |
| Acid value, mg KOH/g | 1.12 |
| Iodine value, gI₂/100 g | 1 |
| Sulfur content, mg kg⁻¹ | <5 |
| Nitrogen content, mg kg⁻¹ | 12 |
| Carbon atoms in aromatic bond, % | <0.3% |
| Mass ratio of I-/n-paraffins | 0.03 |
| C₃₀₊ content, % | 23.1 |
| Olefin content, % | <0.5 |

The dehydrogenation of wax was carried out at 480° C. using the Pd/K-ZSM-22 catalyst of Example 1, applying the same equipment and the same conditions as described in Example 7. The wax is fed to the catalyst bed as a melt. The reactor system differs from the system described in Example 7 in that the pipeline carrying the reactant wax is kept at a temperature above the pour point of the wax, about 100° C. The liquid and/or solid product mixture at room temperature is collected for sufficient time to demonstrate the efficiency of the ethenolysis reaction. The mass of the reactant loaded and the resulting liquid/solid product mixture is measured. From the mass difference, we concluded that less than 1.0 wt % of the reactant was converted to a non-condensable gaseous hydrocarbon product at room temperature.

The olefin content of the liquid product was determined by Raman spectroscopy and ¹H NMR as described in U.S. Pat. No. 7,973,926. The Raman method requires a Raman spectrometer operating in the near infrared frequency range. The first step in determining the olefin content is to record a reference spectrum with a solvent such as toluene. The second step is to record spectra with known olefin-containing hydrocarbon mixtures. The integrated area of the bands characteristic of olefins appearing between 1635 and 1725 cm⁻¹ is divided by the integrated area of the solvent (toluene) spectrum in this frequency range. The ratio as a function of the olefin content gives the calibration equation for the olefin content. The use of the ratio eliminates the error that would result from intensity variations arising from the fluctuations in laser intensity. Calculation of the unknown olefin content: olefin content, tf. %=M×a+B, where M is the slope of the calibration curve, a (area ratio) is the area of the olefin region in the liquid with unknown olefin content divided by the area of the reference spectrum in the same frequency range, B is the intercept of the calibration line.

The olefin content of the product mixture was 15.2 wt %. The product mixture was used as a feedstock in the catalytic ethenolysis reaction without separation. The composition of the product was also determined by ¹H NMR analysis. To prepare the sample, 20 mg of the resulting mixture was dissolved in a carbon disulfide—deuterated chloroform mixture having a volume ratio of 1:1. The spectra showed that the sample contained solely internal alkenes. Hydrogen atoms in the vinyl position relative to internal olefin bonding gave a signal in the spectrum, while those attached to terminal olefin bonding did not (FIG. 5). By comparing the signal intensity of the terminal methyl hydrogens with the signal intensity of the olefinic hydrogens, it was concluded that the sample contains 10 wt % internal olefin.

The product mixture was used as a feedstock in the catalytic ethenolysis reaction without separation.

### EXAMPLE 10. PRODUCTION OF ALPHA-OLEFINS BY ETHENOLYSIS OF DEHYDROGENATED FT WAX

To demonstrate the reaction, the product obtained by dehydrogenation of FT wax as in Example 9 is undertaken to ethenolysis using a pressure-resistant batch reactor under 10 bar ethylene pressure. The homogeneous catalyst BICAAC-Ru according to Example 4 a) and the heterogenized ruthenium complex bis-CAAC/Amberlyst 15 prepared according to Example 5 or BICAAC/ZSM-22 according to Example 4 b) are used as a catalyst. The components of the reaction mixture are measured as described in Example 8. To 1000 mg of the mixture containing high carbon number (>C₂₀) alkanes and olefins (containing 100 mg unsaturated alkenes in the internal position), 2 ml of deuterated toluene and 5 mg of metathesis catalyst were added (M=618.65 g/mol, corresponding to 5 mol % of catalyst per double bond, calculated on an average molar mass of 450 g/mol of olefins). The reaction mixture was stirred at 75° C. for 4 hours under 10 bar ethylene pressure. At the end of the reaction, the mixture was cooled and an emulsion was formed. Of this material, 20 mg was dissolved in a 1:1 vol. mixture of carbon disulfide and deuterated chloroform and the ¹H NMR spectrum of the solution was recorded (FIG. 6). Based on the results of this study, the proportion of terminal olefins was determined for the olefin content of the sample, which could be as high as 95%.

Products—that are in a liquid and gas phases at room temperature—were analyzed by GC-MS. The mass of C₃-C₄ olefins detected in the gas did not exceed 1 wt % of the mass loaded. The maximum of the molecular weight distribution of the liquid was shifted towards lower molecular weights, which is clear evidence of metathesis-type carbon chain shortening.

### EXAMPLE 11. ETHYLENE ISOMET REACTION OF AN OLEFIN MIXTURE USING A HOMOGENEOUS-PHASE METATHESIS AND HOMOGENEOUS-PHASE ISOMERIZATION CATALYST

We used the same experimental equipment and reaction control as in Example 8. 4000 mg of the model compound, 1-octadecene, is weighed into a stirred, pressure-resistant reactor. The metathesis catalyst is added (BICAAC, 0.1 mg, M=618.65 g/mol) in solid state or as stock solution according to Example 4.a. and the catalyst for isomerization (ruthenium hydride complex, RuH(CO)Cl(PPh₃)₃ 3 mg) and 3 ml of toluene solvent. The catalyst of the isomerization is not soluble in most organic solvents at room temperature, only in a 1:1 volume ratio of chloroform-THF. The reactor is then sealed, and the system is flushed with ethylene gas using a suitable fitting. The reaction is performed in excess ethylene in the presence of ethylene at 10 bar pressure at 75° C. for 24 hours. To promote the conversion of olefin to propylene, the partial pressure of ethylene in the gas chamber is kept high, so that the reactor is periodically flushed with fresh ethylene gas or fresh ethylene gas is continuously bubbled through the liquid phase. Samples of the gas and liquid phases of the reactor are taken every 3 hours and analyzed with gas chromatography. It is found that the ISOMET reaction approaches its equilibrium when the concentration of products, mainly propylene, reaches about 30% by volume at the expense of ethylene concentration. The hydrocarbon composition of the gaseous reaction mixture was determined using a gas chromatograph equipped with a flame ionization detector. The amount of propylene formed can be calculated from the chromatographic data.

### EXAMPLE 12. ETHYLENE ISOMET REACTION OF AN OLEFIN MIXTURE USING A HETEROGENIZED METATHESIS AND HOMOGENEOUS-PHASE ISOMERIZATION CATALYST

When testing the activity of the heterogeneous metathesis catalyst, the same experimental setup and reaction procedure is followed as in Example 10. To 2000 mg of the model compound 1-octadecene, 2 ml of hexane and 50 mg of Amberlyst 15-supported BICAAC metathesis catalyst according to Example 5 or 100 mg of aluminum oxide supported BICAAC metathesis catalyst according to Example 6, and homogeneous-phase isomerization catalyst (ruthenium hydride complex, RuH(CO)Cl(PPh₃)₃ 3 mg) are added (in these, the molar mass of the active phase is M=618.65 g/mol, corresponding to approx. 5 mol % catalyst). The reaction mixture is stirred at 75° C. for 24 hours under 10 bar ethylene pressure. At the end of the reaction, the mixture is cooled, and an emulsion is formed. Samples of the gas and liquid phases of the reactor are taken every 3 hours and analyzed with gas chromatography. It is found that the ISOMET reaction approaches its equilibrium when the concentration of products, mainly propylene, reaches about 30% by volume at the expense of ethylene concentration. The hydrocarbon composition of the gaseous reaction mixture was determined using a gas chromatograph equipped with a flame ionization detector. The amount of propylene formed can be calculated from the chromatographic data.

1. A process for preparing linear alpha-olefins, having a carbon number of 3-42, or products containing linear alpha-olefins, having a carbon number of 3-42,
which process comprises the following steps a) and b):
a) paraffin having a carbon number of 11-45, or a paraffin mixture containing paraffins having a carbon number of 11-45 is dehydrogenated using a zeolite-supported heterogeneous Pt, Pd or Ni catalyst, which catalyst is
characterized by that
it contains a medium pore-sized zeolite as a support, in which the zeolite Si/Al ratio is 2-250, and which has Pt, Pd or Ni metal introduced into the zeolite support with a molar amount not exceeding the framework aluminum content of the zeolite support, at a dispersity higher than 10% and in the form of neutral metal atoms or neutral metal nanoparticles;
and/or
it is produced by the following process steps:
(i) a Pt, Pd or Ni salt, preferably Pt, Pd or Ni nitrate, acetate, hydroxide, or a Pt, Pd or Ni amine complex, of a molar amount not exceeding the framework aluminum content of the zeolite, is introduced by ion exchange or impregnation into the Na, K, or ammonium ion form of a medium pore-sized zeolite with a Si/Al ratio of 2-250,
(ii) the material obtained in step (i) is calcined at 300-500° C.
(iii) the material calcined in step (ii) is reduced with hydrogen at 300-500° C.,
(iv) the material reduced in step (iii) is ion-exchanged with a Na or K salt solution, or
it is reacted in solid phase with Na or K salt in an amount equivalent to the lattice aluminum content of the zeolite, and the salt is then decomposed by heat treatment;
and
b) the internal monoolefin content of the mixture obtained in step a) is converted to alpha-olefins,
using a homogeneous or heterogenized homogeneous ruthenium complex metathesis catalyst in ethylene metathesis (ethenolysis) reaction, and/or
using a homogeneous or heterogenized homogeneous ruthenium complex metathesis catalyst in combination with a homogeneous or heterogeneous olefin isomerization catalyst in an isomerization metathesis (ISOMET) reaction, and/or
using a homogeneous or heterogenized homogeneous ruthenium complex metathesis catalyst in combination with a homogeneous or heterogeneous olefin isomerization catalyst in an ethylene isomerization metathesis (ethylene ISOMET) reaction;
or a process comprising the following step b):
b) an internal monoolefin having a carbon number of 11-45, or the internal monoolefin content of a mixture containing an internal monoolefin having a carbon number of 11-45 is converted to alpha-olefins,
using a homogeneous or heterogenized homogeneous ruthenium complex metathesis catalyst in ethylene metathesis (ethenolysis) reaction, and/or
using a homogeneous or heterogenized homogeneous ruthenium complex metathesis catalyst in combination with a homogeneous or heterogeneous olefin isomerization catalyst in an isomerization metathesis (ISOMET) reaction, and/or
using a homogeneous or heterogenized homogeneous ruthenium complex metathesis catalyst in combination with a homogeneous or heterogeneous olefin isomerization catalyst in an ethylene isomerization metathesis (ethylene ISOMET) reaction;
where the ruthenium complex in the homogeneous or heterogenized homogeneous ruthenium complex metathesis catalyst used in step b) is of formula I, II, III or IV, 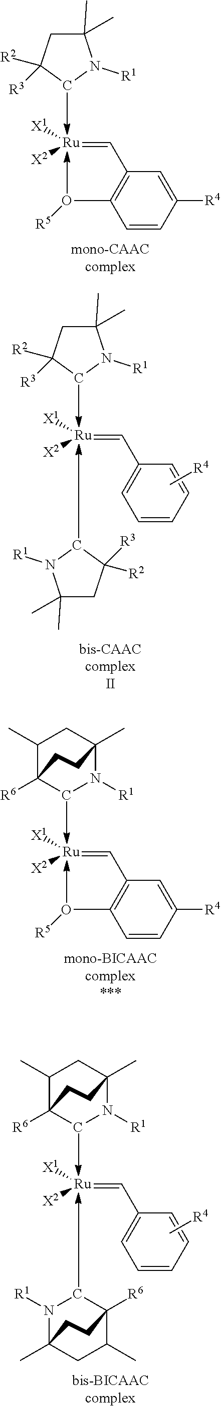
wherein
X¹ and X² are independently halogen, nitrite, nitrate, sulfate, carboxylate, sulfonate, alkoxy, alkylthio, or
X¹ and X² represent a heteroatom linked together via a part of an organic molecule, for example, X¹ and X² represent O and form together through a benzene ring a catecholate group, or X¹ and X² are S and form together through a benzene ring a catecho-ditiolate group;
R¹ represents aryl, heteroaryl or alkyl optionally substituted by one or more substituents, where the substituents are independently selected from alkyl, aryl, heteroaryl, amino, alkylamino, dialkylamino, quaternary ammonium ion, nitro, sulfone, sulfonic acid, sulfonic acid derivative, ketone, carboxylic acid, carboxylic acid derivative, alkoxy, preferably an electron donating group such as dimethylamino or trimethylammonium ion,
with the proviso, that when R¹ is aryl or heteroaryl, in the case of a complex containing a BICAAC ligand of formula (III) or (IV) the substituent in the ortho position relative to the nitrogen atom is an alkyl containing at most 1-2 carbon atom(s);
R² and R³ are independently alkyl, aryl or heteroaryl, which may be optionally substituted by one or more substituents selected from alkyl, aryl, heteroaryl, amino, alkylamino, dialkylamino, quaternary ammonium ion, nitro, sulfone, sulfonic acid, sulfonic acid derivative, ketone, carboxylic acid, carboxylic acid derivative, alkoxy,
preferably an electron-donating group,
or R² and R³ form together with the carbon atom to which they are linked a cycloalkane ring having 3-12 carbon atoms;
R⁴ represents hydrogen, alkyl, nitro, carboxylic acid, carboxylic acid derivative, sulfonic acid, sulfonic acid derivative, aryl, heteroaryl, perfluoroalkyl, perfluoroaryl, sulfone, ketone, preferably an electron-withdrawing group such as perfluoroalkyl or nitro;
R⁵ represents alkyl;
R⁶ represents hydrogen or alkyl, or a group linked via a linker of 1-24 carbon atoms, wherein the group is selected from aryl, heteroaryl, amino, alkylamino, dialkylamino, ketone, ether, hydroxy, thioether, thiol, carboxylic acid derivative, sulfonic acid derivative, silicon-containing group, or a ligand selected from amine, imine, thioether, sulfoxide, phosphine, ether, nitrile and isonitrile, optionally linked to other variable groups of the molecule, for example, in the case of a bis-BICAAC complex of formula IV, to the R⁶ group of the other BICAAC ligand;
with the proviso that at least one of the groups R¹, R², R³ or R⁶ contains an ionic or an ion-forming group selected from a quaternary ammonium ion, amine, alkylamino having 1-6 carbon atoms, dialkylamino having 1-6 carbon atoms, carboxylic acid, carboxylate, sulfonic acid or a sulfonic acid salt group.

2. The process according to claim 1, characterized in that, in the process of preparing the zeolite-supported heterogeneous Pt, Pd or Ni catalyst used in step a),
the ammonium ion form of zeolite is applied in step (i), and/or
in step (iv), the material reduced in step (iii) is reacted in solid phase with Na or K halide, nitrate, acetate, or hydroxide in an amount equivalent to the framework aluminum content of the zeolite, and the salt is then decomposed by heat treatment.

3. The process according to claim 1, characterized in that in step a) the zeolite support for the catalyst is an MFI, TON, or IMF framework-type zeolite, preferably a zeolite of type ZSM-5, ZSM-22, or IM-5, most preferably ZSM-22.

4. The process according to claim 1, characterized that in step a) a Pt/Na-ZSM-22 catalyst is used.

5. The process according to claim 1, characterized in that in step a) the zeolite support of the catalyst has a Si/Al ratio of 5-150, most preferably of 10-80.

6. The process according to claim 1, characterized in that step a) is performed using a catalytic tubular reactor at a hydrogen overpressure of 0.5-1.0 bar, at 350-500° C., applying a paraffin liquid space velocity (LHSV) of 5-25 h⁻¹, preferably of 15-25 h⁻¹, most preferably of 20 h⁻¹, a hydrogen gas space velocity (GHSV) of 8,000-15,000 h⁻¹, preferably of 10,000-15,000 h⁻¹, most preferably of 12,000 h⁻¹.

7. The process according to claim 1, characterized in that bioparaffin is used as the starting material of step a).

8. The process according to claim 1, wherein in the formula of I, II, III or IV,
X¹ and X² are independently halogen, nitrite, nitrate, sulfate, carboxylate, sulfonate, alkoxy having 1-6 carbon atoms or alkylthio having 1-6 carbon atoms, or X¹ and X² are heteroatoms linked together via a part of an organic molecule, for example, X¹ and X² are O and form together through a benzene ring a catecholate group, or X¹ and X² are S and form together through a benzene ring a catecho-ditiolate group;
R¹ represents aryl, heteroaryl or alkyl optionally substituted by one or more substituents, where the substituents are independently selected from alkyl, aryl, heteroaryl, amino, alkylamino having 1-6 carbon atoms, dialkylamino having 1-6 carbon atoms, quaternary ammonium ion, nitro, sulfone containing an alkyl group having 1-6 carbon atoms, sulfonic acid, sulfonic acid derivative, ketone containing an alkyl group having 1-6 carbon atoms, carboxylic acid, carboxylic acid derivative, alkoxy having 1-6 carbon atoms, preferably an electron-donating group such as dimethylamino or trimethylammonium ion with the proviso, that when R¹ is aryl or heteroaryl, in the case of a complex containing a BICAAC ligand of formula (III) or (IV) the substituent in the ortho position relative to the nitrogen atom is an alkyl containing at most 1-2 carbon atom(s);
R² and R³ are independently alkyl, aryl or heteroaryl, which may be optionally substituted by one or more substituents selected from alkyl, aryl, heteroaryl, amino, alkylamino having 1-6 carbon atoms, dialkylamino having 1-6 carbon atoms, quaternary ammonium ion, nitro, sulfone containing an alkyl group having 1-6 carbon atoms, sulfonic acid, sulfonic acid derivative, ketone containing an alkyl having 1-6 carbon atoms, carboxylic acid, carboxylic acid derivative, alkoxy having 1-6 carbon atoms, a preferably electron-donating group, or R² and R³ form together with the carbon atom to which they are linked a cycloalkane ring having 3-12 carbon atoms;
R⁴ represents hydrogen, alkyl, nitro, carboxylic acid, carboxylic acid derivative, sulfonic acid, sulfonic acid derivative, aryl, heteroaryl, perfluoroalkyl, perfluoroaryl, sulfone containing an alkyl group having 1-6 carbon atoms, ketone containing alkyl group having 1-6 carbon atoms, preferably an electron-withdrawing group such as perfluoroalkyl or nitro;
R⁵ represents alkyl;
R⁶ represents hydrogen or alkyl, or a group linked via a linker of 1-24 carbon atoms, wherein the group is selected from aryl, heteroaryl, amino, alkylamino having 1-6 carbon atoms, dialkylamino having 1-6 carbon atoms, ketone containing an alkyl group having 1-6 carbon atoms, ether containing an alkyl group having 1-6 carbon atoms, alcohol, thioether containing an alkyl groups having 1-6 carbon atoms, thiol, a carboxylic acid derivative, a sulfonic acid derivative, a silicon-containing group, or a ligand selected from amine, imine, sulfide, sulfoxide, phosphine, ether, nitrile and isonitrile, optionally attached to other variable groups of the molecule, for example, in the case of a bis-BICAAC complex of formula IV, to the R⁶ group of the other BICAAC ligand;
with the proviso that at least one of the groups R¹, R², R³ or R⁶ contains an ionic or an ion-forming group.

9. The process according to claim 1, wherein in the formula of I, II, III or IV
X¹ and X² represents halogen, preferably chlorine;
R¹ represents a phenyl substituted by one or more substituents selected from alkyl, amino, alkylamino having 1-6 carbon atoms, dialkylamino having 1-6 carbon atoms, quaternary ammonium ion, preferably 2,6-dimethyl-4-dimethylamino-phenyl or 2,6-dimethyl-4-trimethylammonium-phenyl;
R² and R³ are independently alkyl or aryl,
R⁴ represents hydrogen;
R⁵ represents alkyl;
R⁶ represents alkyl;
with the proviso that R¹ contains an ionic or an ion-forming group.

10. The process according to claim 1, characterized in that the ruthenium complex in the ruthenium complex metathesis catalyst is of formula III and/or IV.

11. The process according to claim 1, wherein in step b) as homogeneous or heterogenized homogeneous ruthenium complex metathesis catalyst one or more complexes selected from the following is used:

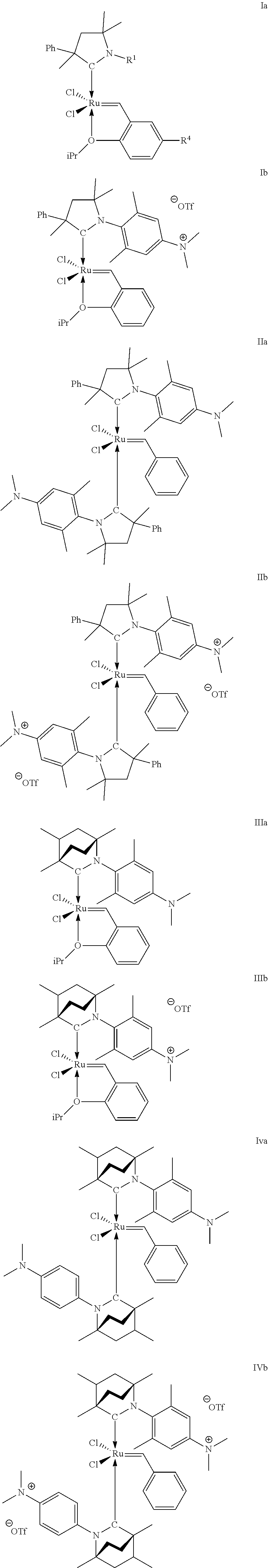

12. The process according to claim 1, characterized in that in step b) the homogeneous or heterogenized homogeneous ruthenium complex catalyst is used in combination with a homogeneous or heterogeneous olefin double bond isomerization catalyst and thus the mixture containing internal monoolefins is converted by ethylene isomerization metathesis (ethylene ISOMET) in the presence of excess ethylene, to propylene or to mostly propylene-containing alpha-monoolefin homologues with shortening carbon chains as the reaction proceeds.

13. The process according to claim 1, characterized in that in step b) a heterogenized homogeneous ruthenium complex metathesis catalyst is used, which is produced in a process involving ion exchange or ionic bond forming between the ruthenium complex and zeolite, preferably H-, K-, or Na-ZSM-22 zeolite or a poly(styrene divinylbenzene) sulfonic acid or its alkali metal salt, preferably Amberlyst 15 resin, or produced by adsorption on a high specific surface area oxide support, including γ-aluminum oxide.

14. The process according to claim 1, characterized in that in step b) as olefin double bond isomerization catalyst, a homogenous catalyst and/or a solid acid catalyst, preferably a homogeneous RuH(CO)Cl(PPh₃)₃ catalyst, and/or a zeolite, preferably H-Beta, H—Y or USY zeolite, and/or a polymer functionalized with an acid group, preferably Amberlyst 15 resin, solid acid catalyst, most preferably a heterogenized bifunctional solid acid-supported ruthenium complex catalyst is used.

15. The process according to claim 13, characterized in that step b) is performed in a continuous-flow tubular reactor at 80-100° C., at a hydrocarbon fluid space velocity (LHSV) of 0.1-3.0 h⁻¹, preferably at 0.1-1.0 h⁻¹, most preferably at 0.1-0.5 h⁻¹ hydrocarbon fluid space velocity, at 250-450 h⁻¹, preferably at 300-350 h⁻¹ ethylene space velocity (GHSV), at 1.0-10.0 bar ethylene overpressure, preferably at 2.0-3.0 bar pressure at 2-10 times molar excess ethylene, preferably at 7-10 times, most preferably at 10 times excess ethylene.

16. The process according to claim 1, characterized in that the paraffin/olefin mixture obtained in step a) is converted in step b) by ethylene in a metathesis (ethenolysis) reaction in a low-boiling-point solvent, preferably in hexane or heptane, applying a reaction temperature of 15-50° C., preferably room temperature.

17. The process according to claim 1, characterized in that after step b) the ethylene-rich gaseous product mixture is recycled to step b).

18. The process according to claim 1, characterized in that the liquid product mixture of step b) is fractionated and the long-chain hydrocarbons remaining in the bottom product, essentially paraffins, are recycled to step a).

19. The process according to claim 1, characterized in that in step b) the solvent of the ethenolysis, performed in a solvent, is separated from the product mixture and recycled to the ethenolysis process.

20. The ruthenium complex of formula III or IV, 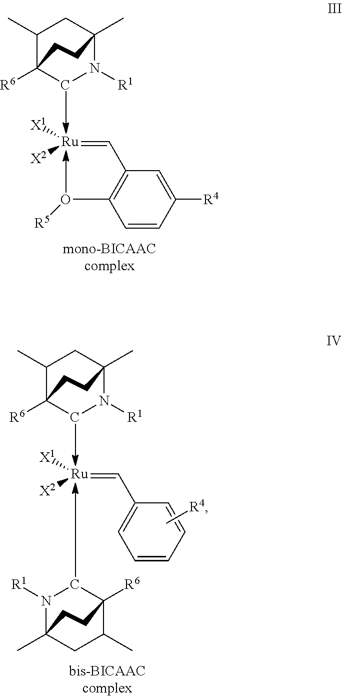
wherein,
X¹ and X² are independently halogen, nitrite, nitrate, sulfate, carboxylate, sulfonate, alkoxy or alkylthio, or
X¹ and X² are heteroatoms linked together via a part of an organic molecule, for example, X¹ and X² are O and form together through a benzene ring a catecholate group, or X¹ and X² are S and form together through a benzene ring a catecho-ditiolate group;
R¹ represents aryl, heteroaryl or alkyl optionally substituted by one or more substituents, where the substituents are independently selected from alkyl, aryl, heteroaryl, amino, alkylamino, dialkylamino, quaternary ammonium ion, nitro, sulfone, sulfonic acid, sulfonic acid derivative, ketone, carboxylic acid, carboxylic acid derivative, alkoxy, preferably an electron donating group such as dimethylamino or trimethylammonium ion, with the proviso, that when R¹ is aryl or heteroaryl, the substituent in the ortho position relative to the nitrogen atom is an alkyl containing at most 1-2 carbon atom(s);
R⁴ represents hydrogen, alkyl, nitro, carboxylic acid, carboxylic acid derivative, sulfonic acid, sulfonic acid derivative, aryl, heteroaryl, perfluoroalkyl, perfluoroaryl, sulfone, ketone, preferably an electron-withdrawing group such as perfluoroalkyl or nitro;
R⁵ represents alkyl;
R⁶ represents hydrogen or alkyl, or a group linked via a linker of 1-24 carbon atoms, wherein the group is selected from aryl, heteroaryl, amino, alkylamino, dialkylamino, ketone, ether, hydroxy, thioether, thiol, carboxylic acid derivative, sulfonic acid derivative, silicon-containing group or a group selected from amine, imine, sulfide, sulfoxide, phosphine, ether, nitrile and isonitrile, optionally linked to other variable groups of the molecule, for example, in the case of a bis-BICAAC complex of formula IV, to the R⁶ group of the other BICAAC ligand;
with the proviso that at least one of the groups R¹ or R⁶ contains at least one ionic or ion-forming group selected from a quaternary ammonium ion, amine, alkylamino having 1-6 carbon atoms, dialkylamino having 1-6 carbon atoms, carboxylic acid, carboxylate, sulfonic acid or a sulfonic acid salt group.

21. The ruthenium complex according to claim 20, wherein
X¹ and X² are independently halogen, nitrite, nitrate, sulfate, carboxylate, sulfonate, alkoxy having 1-6 carbon atoms or alkylthio having 1-6 carbon atoms, or X¹ and X² are heteroatoms linked together via a part of an organic molecule, for example, X¹ and X² are O and form together through a benzene ring a catecholate group, or X¹ and X² are S and form together through a benzene ring a catecho-ditiolate group;
R¹ represents aryl, heteroaryl or alkyl optionally substituted by 1 or more substituents, where the substituents are independently selected from alkyl, aryl, heteroaryl, amino, alkylamino having 1-6 carbon atoms, dialkylamino having 1-6 carbon atoms, quaternary ammonium ion, nitro, sulfone containing an alkyl group having 1-6 carbon atoms, sulfonic acid, sulfonic acid derivative, ketone containing an alkyl group having 1-6 carbon atoms, carboxylic acid, carboxylic acid derivative, alkoxy having 1-6 carbon atoms, preferably an electron-donating group such as dimethylamino or trimethylammonium ion, with the proviso, that when R¹ is aryl or heteroaryl, the substituent in the ortho position relative to the nitrogen atom is an alkyl containing at most 1-2 carbon atom(s);
R⁴ represents hydrogen, alkyl, nitro, carboxylic acid, carboxylic acid derivative, sulfonic acid, sulfonic acid derivative, aryl, heteroaryl, perfluoroalkyl, perfluoroaryl, sulfone containing an alkyl group having 1-6 carbon atoms, ketone containing an alkyl group having 1-6 carbon atoms, preferably an electron-withdrawing group such as perfluoroalkyl or nitro;
R⁵ represents alkyl;
R⁶ represents hydrogen or alkyl, or a group linked via a linker of 1-24 carbon atoms, wherein the group is selected from aryl, heteroaryl, amino, alkylamino having 1-6 carbon atoms, dialkylamino having 1-6 carbon atoms, ketone containing an alkyl group having 1-6 carbon atoms, ether containing an alkyl groups having 1-6 carbon atoms, alcohol, thioether containing an alkyl group having 1-6 carbon atoms, thiol, a carboxylic acid derivative, a sulfonic acid derivative, a silicon-containing group, or a group selected from amine, imine, sulfide, sulfoxide, phosphine, ether, nitrile and isonitrile, optionally attached to other variable groups of the molecule, for example, in the case of a bis-BICAAC complex of formula IV, to the R⁶ group of the other BICAAC ligand;
with the proviso that at least one of the groups R¹ or R⁶ contains an ionic or an ion-forming group.

22. The ruthenium complex of claim 20, wherein
X¹ and X² are independently halogen, nitrite, nitrate, sulfate, carboxylate, sulfonate, alkoxy having 1-6 carbon atoms or alkylthio having 1-6 carbon atoms;
R¹ represents aryl, heteroaryl optionally substituted by 1 or more substituents, where the substituents are independently selected from alkyl, aryl, heteroaryl, amino, alkylamino having 1-6 carbon atoms, dialkylamino having 1-6 carbon atoms, quaternary ammonium ion, nitro, sulfone containing an alkyl group having 1-6 carbon atoms, sulfonic acid, sulfonic acid derivative, ketone containing an alkyl group having 1-6 carbon atoms, carboxylic acid, carboxylic acid derivative, alkoxy having 1-6 carbon atoms, preferably an electron-donating group such as dimethylamino or trimethylammonium ion, with the proviso, that the substituent in the ortho position relative to the nitrogen atom is an alkyl containing at most 1-2 carbon atom(s);
R⁴ represents hydrogen, alkyl, nitro, carboxylic acid, carboxylic acid derivative, sulfonic acid, sulfonic acid derivative, aryl, heteroaryl, perfluoroalkyl, perfluoroaryl, sulfone containing an alkyl group having 1-6 carbon atoms, ketone containing an alkyl group having-6 carbon atoms, preferably hydrogen or an electron-withdrawing group such as perfluoroalkyl or nitro;
R⁵ represents alkyl;
R⁶ represents hydrogen or alkyl;
with the proviso that R¹ contains an ionic or an ion-forming group.

23. The ruthenium complex of claim 20, wherein
X¹ and X² represent halogen, preferably chlorine;
R¹ represents phenyl optionally substituted by one or more substituents selected from alkyl, amino, alkylamino having 1-6 carbon atoms, dialkylamino having 1-6 carbon atoms, quaternary ammonium ions, preferably 2,6-dimethyl-4-dimethylamino-phenyl or 2,6-dimethyl-4-trimethylammonium-phenyl;
R⁴ represents hydrogen;
R⁵ represents alkyl;
R⁶ represents alkyl
with the proviso that R¹ contains an ionic or an ion-forming group.

24. The ruthenium complex of claim 20, which is selected from the following.

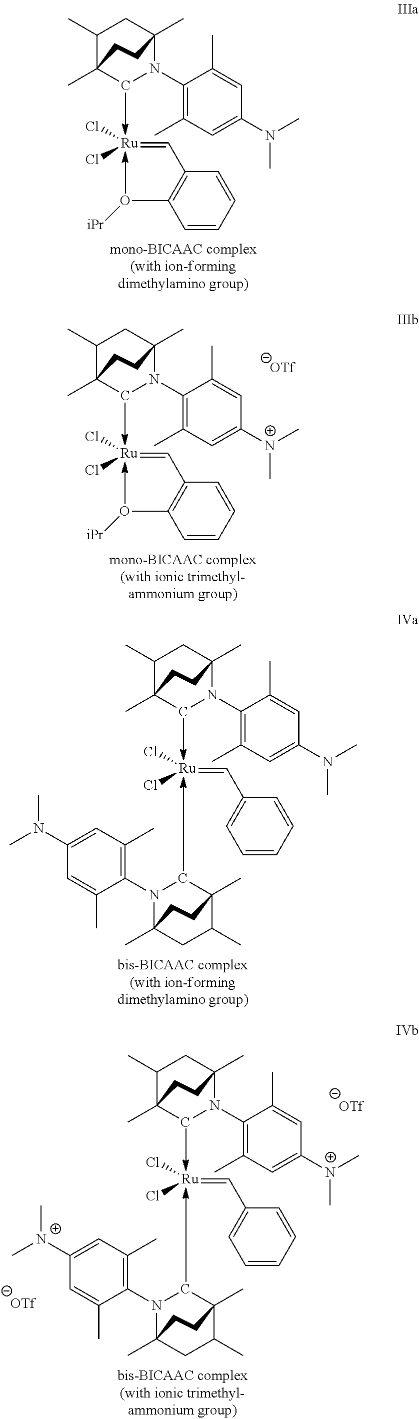

25. A catalyst in the metathesis reaction of olefins comprising a ruthenium complex according to claim 20 in its homogeneous or heterogenized form.

26. The heterogenized homogeneous ruthenium complex metathesis catalyst prepared in a process involving ion exchange or ionic bond formation between a ruthenium complex as defined in claim 1 and a zeolite, preferably H-, K- or Na-ZSM-22 zeolite, or a poly(styrene-divinylbenzene)-sulfonic acid or its alkali metal salt, preferably Amberlyst 15 resin; or
by adsorption of a ruthenium complex as defined in claim 1 on a high specific surface area oxide support, including on γ-aluminum oxide.

27. A zeolite-supported Pt, Pd or Ni catalyst, characterized by that
it contains a medium pore-sized zeolite as a support, in which the zeolite Si/Al ratio is 2-250, and which has Pt, Pd or Ni metal introduced into the zeolite support with a molar amount not exceeding the framework aluminum content of the zeolite support, at a dispersity higher than 10% and in the form of neutral metal atoms or neutral metal nanoparticles;
and/or
it is produced by the following process steps:
(i) a Pt, Pd or Ni salt, preferably Pt, Pd or Ni nitrate, acetate, hydroxide, or a Pt, Pd or Ni amine complex, of a molar amount not exceeding the framework aluminum content of the zeolite, is introduced by ion exchange or impregnation into the Na, K, or ammonium ion form of a medium pore-sized zeolite with a Si/Al ratio of 2-250,
(ii) the material obtained in step (i) is calcined at 300-500° C.
(iii) the material calcined in step (ii) is reduced with hydrogen at 300-500° C.,
(iv) the material reduced in step (iii) is ion-exchanged with a Na or K salt solution, or
it is reacted in solid phase with Na or K salt in an amount equivalent to the lattice aluminum content of the zeolite, and the salt is then decomposed by heat treatment.
